(19) United States
(12) Patent Application Publication
(10) Pub. No.: US 2007/0015789 A1
(43) Pub. Date: Jan. 18, 2007

(54) Novel methods using aminobenzoic acid compounds

(76) Inventors: Jay Barth, Teaneck, NJ (US); Kathleen Lomax, Teaneck, NJ (US); Scott Fields, Teaneck, NJ (US); Mindell Seidlin, Teaneck, NJ (US)

Correspondence Address: VENABLE LLP, P.O. BOX 34385, WASHINGTON, DC, 20043-9998, US

(21) Appl. No.: 11/332,711

(22) Filed: Jan. 11, 2006

Related U.S. Application Data
(63) Continuation of application No. PCT/US04/21858, filed on Jul. 9, 2004.
(60) Provisional application No. 60/486,198, filed on Jul. 11, 2003.

(51) Int. Cl. A61K 31/46

(52) U.S. Cl. 514/304

(57) ABSTRACT

The invention provides safe and effective methods for treating and/or preventing gastrointestinal disorders, psychiatric disorders, learning disabilities, Tourette's syndrome, obesity, epilepsy, post-menopausal syndrome, pre-menstrual syndrome, asthma, laryngitis and/or migraines by administering to a patient in need thereof at least one aminobenzoic acid compound. The aminobenzoic acid compound can optionally be administered in combination with other medications that are useful for the disease being treated.

### RELATED APPLICATIONS

This application is a continuation of PCT/US04/021858 filed Jul. 9, 2004 which claims priority to U.S. Application No. 60/486,198 filed Jul. 11, 2003, the disclosures of which are incorporated by reference herein in their entirety.

### FIELD OF THE INVENTION

The invention provides safe and effective methods for treating and preventing diseases and disorders by administering to patients in need thereof one or more aminobenzoic acid compounds.

### BACKGROUND OF THE INVENTION

The proportion of patients with complaints of unknown etiology such as abdominal full-consciousness, heartburn, nausea and vomiting among those with gastrointestinal diseases has recently increased steadily and now reaches 60% or above. Most of these type complaints are caused by a functional abnormality of the digestive tract. For example, it is known that a patient with epigastric unidentified complaints such as chronic gastritis is in a state of delayed gastric emptying, while a patient with hypogastric unidentified complaints such as irritable bowel syndrome including abnormal evacuation and abdominal pain as main symptoms is in a state of intestinal hyperanakinezia.

It has been established that stress and anxiety are causative of complaints of unknown etiology, and in this regard, these complaints are one of many modern diseases. The gastrointestinal complaints of unknown etiology are ameliorated by three classes of compounds—dopamine antagonists, musculotropic agents for regulating the movement of smooth muscles and acetylcholine release accelerators. Adverse reactions however, usually accompany these drugs. For instance, dopamine antagonists cause potent adverse reactions such as parkinsonism, so that they must be used carefully. Musculotropic agents used for regulating the movement of smooth muscles cause adverse reactions such as constipation. Acetylcholine release accelerators do not effectively act as an antiemetic or ataractic and are thus not effective.

Due to the ineffectiveness and adverse side effects of the above mentioned compounds, the present inventors set out in a number of studies to develop a class of drugs which is efficacious in treating all types of patients with gastrointestinal complaints of unknown etiology or vague gastrointestinal complaints. These compounds need to be effective without the associated adverse reactions and they should also exhibit a depressant activity against anxiety, a symptom which is nearly always present as one of the background factors of such patients.

As a result of the studies undertaken, it has been concluded that the above requirements can be fulfilled by a drug exhibiting both a serotonin (hereinafter abbreviated to “5HT₃”) antagonism and an acetylcholine (hereinafter abbreviated to “ACh”) release accelerating activity. Therefore, further studies have been conducted in order to find a compound exhibiting both the aforementioned attributes in an activity ratio that is well-balanced. Indeed, both attributes can be produced by using an aminobenzoic acid compound of the invention. There is a need in the art for new and improved treatments for gastrointestinal disorders. The invention is directed to these, as well as other, important ends.

### SUMMARY OF THE INVENTION

The invention provides safe and effective methods for treating and/or preventing gastrointestinal disorders, psychiatric disorders, learning disabilities, Tourette's syndrome, obesity, epilepsy, post-menopausal syndrome, pre-menstrual syndrome, asthma, laryngitis and/or migraines by administering to a patient in need thereof at least one aminobenzoic acid compound. The aminobenzoic acid compound can optionally be administered in combination with other medications that are useful for the disease being treated.

The invention is described in more detail below.

### DETAILED DESCRIPTION OF THE INVENTION

The invention provides methods for treating and preventing gastrointestinal disorders in a patient in need thereof by administering a therapeutically effective amount of at least one aminobenzoic acid compound. The gastrointestinal disorder can be any in the art, and can include gastrointestinal disorders of the upper and/or lower gastrointestinal tracts. Exemplary gastrointestinal disorders include ulcers, post-operative aspiration, dyspepsia, acute gastrointestinal bleeding, lower esophageal mucosal rings, esophageal strictures, esophageal dismotility, hiatal hernia, achlasia, irritable bowel syndrome, Barrett's esophagus, gastroparesis, gastrointestinal motility disorders, diverticulosis, diverticulitis, malabsorption syndromes, gastroesophageal reflux disease (GERD), problems caused by esophageal bypass surgery, belching, eructation, flatulence, diarrhea, inflammatory bowel disease, infectious enteritis, idiopathic gastric acid hypersecretion, gastritis, constipation, colic, vomiting, nausea, motion sickness, gastrointestinal injuries, esophageal injuries, gastric mucosal injuries, short bowel syndrome, bowel dysfunctions, early satiety, abdominal pain, abdominal bloating, sour stomach, radiation-induced injury to the gastrointestinal tract, gastrointestinal disorders induced by medications, chronic sore throat, noncardiac chest pains, coughing, dysphagia, Shwachman syndrome, decreased gastric mucin production, iron deficiency anemia, decreased nasal airflow, pancreatitis, cystic fibrosis and the like.

In one embodiment, the gastrointestinal disorders are ulcers, post-operative aspiration, dyspepsia, acute gastrointestinal bleeding, lower esophageal mucosal rings, esophageal strictures, esophageal dismotility, hiatal hernia, achlasia, Barrett's esophagus, gastrointestinal motility disorders, diverticulosis, diverticulitis, malabsorption syndromes, gastroesophageal reflux disease (GERD), problems caused by esophageal by-pass surgery, belching, eructation, flatulence, diarrhea, inflammatory bowel disease, infectious enteritis, idiopathic gastric acid hypersecretion, gastritis, constipation, colic, nausea, motion sickness, gastrointestinal injuries, esophageal injuries, gastric mucosal injuries, short bowel syndrome, bowel dysfunctions, early satiety, abdominal pain, abdominal bloating, sour stomach, radiation-induced injury to the gastrointestinal tract, gastrointestinal disorders induced by medications, chronic sore throat, noncardiac chest pains, coughing, dysphagia, Shwachman syndrome, decreased gastric mucin production, iron deficiency anemia, decreased nasal airflow, pancreatitis, or cystic fibrosis.

In another embodiment, the gastrointestinal disorders are ulcers, post-operative aspiration, dyspepsia, acute gastrointestinal bleeding, diverticulosis, diverticulitis, malabsorption syndromes, gastroesophageal reflux disease (GERD), problems caused by esophageal by-pass surgery, inflammatory bowel disease, infectious enteritis, idiopathic gastric acid hypersecretion, gastritis, colic, motion sickness, gastrointestinal injuries, esophageal injuries, gastric mucosal injuries, short bowel syndrome, bowel dysfunctions, radiation-induced injury to the gastrointestinal tract, gastrointestinal disorders induced by medications, chronic sore throat, noncardiac chest pains, coughing, dysphagia, Shwachman syndrome, decreased gastric mucin production, iron deficiency anemia, decreased nasal airflow, pancreatitis, or cystic fibrosis. In another embodiment, the gastrointestinal disorders is gastroesophageal reflux disease (GERD). In another embodiment, the gastrointestinal disorder is an ulcer.

The methods for treating and/or preventing the gastrointestinal disorders described herein can comprise administering at least one aminobenzoic acid compound and at least one proton pump inhibitor. The aminobenzoic acid compound and the proton pump inhibitor can be administered separately or in the form of a composition. Exemplary proton pump inhibitors include rabeprazole, omeprazole, lansoprazole, esomeprazole, pantoprazole, leminoprazole, timoprazole, tenatoprazole, disulprazole, RO 18-5362, IY 81149 and the like. In other embodiments, the invention provides methods for treating and/or preventing the gastrointestinal disorders described herein by administering at least one aminobenzoic acid compound, at least one proton pump inhibitor, and, optionally, one or more compounds selected from histamine antagonists, antacids, bismuth compounds, VIP (or analogs or fragments thereof), secretin (or analogs or fragments thereof), sucralfate, cisapride, misoprostol, a vitamin supplement, and a mixture of two or more thereof.

The method for treating and/or preventing the gastrointestinal disorders described herein can comprise administering at least one aminobenzoic acid compound and at least one histamine antagonist. The aminobenzoic acid compound and the histamine antagonist can be administered separately or in the form of a composition. Exemplary histamine antagonists include ranitidine, ranitidine/bismuth citrate, cimetidine, famotidine, nizatidine, roxatidine, ebrotidine, burimamide, tiotidine, metiamide, oxmetidine, famotidine/calcium carbonate/magnesium hydroxide, and the like. In other embodiments, the invention provides methods for treating and/or preventing the gastrointestinal disorders described herein by administering at least one aminobenzoic acid compound, at least one histamine antagonist, and, optionally, one or more compounds selected from proton pump inhibitors, antacids, bismuth compounds, VIP (or analogs or fragments thereof), secretin (or analogs or fragments thereof), sucralfate, cisapride, misoprostol, a vitamin supplement, and a mixture of two or more thereof.

The method for treating and/or preventing the gastrointestinal disorders described herein can comprise administering at least one aminobenzoic acid compound and at least one antacid. The aminobenzoic acid compound and the antacid can be administered separately or in the form of a composition. Any antacid can be used in the compositions and methods of the invention. Exemplary antacids include famotidine/calcium carbonate/magnesium hydroxide, calcium carbonate/magnesium hydroxide, aluminum hydroxide, magnesium hydroxide, magnesium carbonate, magnesium oxide, calcium carbonate, calcium carbonate/simethicone, aluminum hydroxide/magnesium hydroxide/simethicone, simethicone, and the like. In other embodiments, the invention provides methods for treating and/or preventing the gastrointestinal disorders described herein by administering at least one aminobenzoic acid compound, at least one antacid, and, optionally, one or more compounds selected from proton pump inhibitors, histamine antagonists, bismuth compounds, VIP (or analogs or fragments thereof), secretin (or analogs or fragments thereof), sucralfate, cisapride, misoprostol, a vitamin supplement, and a mixture of two or more thereof.

The method for treating and/or preventing the gastrointestinal disorders described herein can comprise administering at least one aminobenzoic acid compound and at least one bismuth compound. The aminobenzoic acid compound and the bismuth compound can be administered separately or in the form of a composition. Any bismuth compound can be used in the compositions and methods of the invention. Exemplary bismuth compounds include bismuth citrate, bismuth salicylate, bismuth tartaric acid, and the like. In other embodiments, the invention provides methods for treating and/or preventing the gastrointestinal disorders described herein by administering at least one aminobenzoic acid compound, at least one bismuth compound, and, optionally, one or more compounds selected from proton pump inhibitors, histamine antagonists, antacids, VIP (or analogs or fragments thereof), secretin (or analogs or fragments thereof), sucralfate, cisapride, misoprostol, a vitamin supplement, and a mixture of two or more thereof.

The method for treating and/or preventing the gastrointestinal disorders described herein can comprise administering at least one aminobenzoic acid compound and at least one compound selected from the group consisting of sucralfate, cisapride and misoprostol. The aminobenzoic acid compound and the at least one compound selected from the group consisting of sucralfate, cisapride and misoprostol can be administered separately or in the form of a composition. In other embodiments, the invention provides methods for treating and/or preventing the gastrointestinal disorders described herein by administering at least one aminobenzoic acid compound, at least one compound selected from the group consisting of sucralfate, cisapride, and misoprostol, and, optionally, one or more compounds selected from proton pump inhibitors, histamine antagonists, antacids, bismuth compounds, VIP (or analogs or fragments thereof), secretin (or analogs or fragments thereof), a vitamin supplement, and a mixture of two or more thereof.

The method for treating and/or preventing the gastrointestinal disorders described herein can comprise administering at least one aminobenzoic acid compound and at least one compound selected from VIP, secretin, and/or fragments or analogs thereof. The aminobenzoic acid derivative and at least one compound selected from VIP, secretin, and/or fragments or analogs thereof can be administered separately or in the form of a composition. In other embodiments, the invention provides methods for treating and/or preventing the gastrointestinal disorders described herein by administering at least one aminobenzoic acid compound, at least one compound selected from VIP, secretin, and/or fragments or analogs thereof, and, optionally, one or more compounds selected from proton pump inhibitors, histamine antagonists, antacids, bismuth compounds, sucralfate, cisapride, misoprostol, a vitamin supplement, and a mixture of two or more thereof.

The method for treating and/or preventing the gastrointestinal disorders described herein can comprise administering at least one aminobenzoic acid compound and a vitamin supplement. The vitamin supplement is preferably an iron supplement and/or a calcium supplement. Alternatively, the vitamin supplement contains at least an iron supplement and/or a calcium supplement. The aminobenzoic acid compound and the vitamin supplement can be administered separately or as components of the same composition. In other embodiments, the invention provides methods for treating and/or preventing the gastrointestinal disorders described herein by administering at least one aminobenzoic acid compound, at least one vitamin supplement and, optionally, on or more compounds selected from proton pump inhibitors, histamine antagonists, antacids, bismuth compounds, sucralfate, cisapride, misoprostol, VIP (or analogs or fragments thereof), secretin (or analogs or fragments thereof), and a mixture of two or more thereof.

The invention provides methods for treating and preventing ulcers in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. “Ulcers” include peptic ulcers, bleeding peptic ulcers, stress ulcers, stomal ulcers, refractory ulcers, ICU-induced ulcers, esophageal ulcers, Zollinger-Ellison syndrome, post-operative ulcers and the like. “Peptic ulcers” include gastric ulcers and duodenal ulcers. The ulcers can be associated with H. pylori. Stress ulcers are clinically distinct from peptic ulcers. Patients with stress ulcers often have multiple lesions in the acid-secreting portion of the stomach, in the antrum and/or the duodenum, and the lesions may be bleeding. Stress ulcers may be present in patients with severe injuries, burns, infections and/or shock. Bleeding peptic ulcers may be duodenal or gastric, or stomal ulcers. Refractory ulcers can be peptic ulcers (e.g., gastric ulcers and duodenal ulcers), and can be present in infants, children or adults. Refractory ulcers are generally defined as ulcers that fail to completely heal after daily treatment with 1 gram of cimetidine for three months. Zollinger-Ellison syndrome refers to ulcer disease of the upper gastrointestinal tract, marked increases in gastric acid secretion, and/or nonbeta islet cell tumors of the pancreas. Post-operative ulcers can occur as a result of post-operative aspiration. Patients in the intensive care unit (ICU) often suffer from a high incidence of acute life-threatening upper gastrointestinal bleeding, such as that caused by stress ulceration. In one embodiment, the invention provides methods for treating and preventing peptic ulcers in infants and children by administering a therapeutically effective amount of at least aminobenzoic acid compound to a patient who is an infant or child. The term “infants” includes neonates, and the term “children” includes adolescents. In another embodiment, the invention provides methods for treating and preventing ulcers (e.g., esophageal ulcers) in immunocompromised patients by administering a therapeutically effective amount of at least one of aminobenzoic acid compound.

The invention provides methods for treating and preventing post-operative aspiration in a patient in need thereof by administering a therapeutically effective amount of at least one aminobenzoic acid compound. Administering at least one aminobenzoic acid compound prior to surgery will reduce gastric pH, which will reduce or eliminate the occurrence of post-operative aspiration or will reduce or eliminate the likelihood of post-operative ulcers occurring as a result of post-operative aspiration. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing dyspepsia in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Dyspepsia refers to upper abdominal pain or discomfort and can also include symptoms of nausea, early satiety and bloating. Dyspepsia can be episodic or chronic.

The invention provides methods for treating and preventing acute gastrointestinal bleeding in a patient in need thereof by administering a therapeutically effective amount of at least one of aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Gastrointestinal bleeding is a general term referring to bleeding from anywhere in the gastrointestinal tract. Many cases are due to peptic ulcers, but other causes are esophageal and/or intestinal bleeding. Bleeding is a potential complication of peptic ulcers, and is often associated with more severe ulcers.

The invention provides methods for treating and preventing lower esophageal mucosal rings in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing esophageal strictures by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing esophageal dismotility by administering to a patient in need thereof a therapeutically effective amount of at least one aminobenzoic acid compound. The esophageal dismotility can be caused, for example, by myasthenia gravis. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastroparesis in a patient in need thereof by administering a therapeutically effective amount of at least one aminobenzoic acid compound. Gastroparesis is delayed gastric emptying of either solids or liquids, and is accompanied by symptoms of postprandial nausea, epigastric pain/burning, bloating, early satiety, excessive eructation, anorexia and/or vomiting. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating patients who have had esophageal bypass surgery by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing a hiatal hernia by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. A hiatal hernia occurs when the upper part of the stomach moves up into the chest through a small opening in the diaphragm, e.g., diaphragmatic hiatus. The hiatal hernia can result in retention of acid and other contents above the opening which reflux into the esophagus.

The invention describes methods for treating and preventing Barrett's esophagus in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Barrett's esophagus is a condition in which the stratified squamous epithelium of the esophagus is replaced by a columnar epithelium with malignant potention.

The invention provides methods for treating and preventing achlasia by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing irritable bowel syndrome by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Irritable bowel syndrome is a common gastrointestinal disease characterized by three clinical variants: (i) chronic abdominal pain and constipation, (ii) chronic intermittent diarrhea, often without pain, and (iii) alternating constipation and diarrhea, with or without abdominal pain.

In other embodiments, the invention provides methods for treating and preventing belching, eructation and/or flatulence by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastrointestinal motility disorders by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing diverticulosis by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing diverticulitis by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastroesophageal reflux disease (GERD) in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. GERD occurs when stomach acid moves in the wrong direction, flowing back up (reflux) into the esophagus causing one or more symptoms of heartburn, coughing, wheezing, hoarseness, regurgitation, epigastric pain, dysphagia, and chest pain. Over time, reflux of acid can erode the lining of the esophagus, leading to inflammation and ulcers, a condition called erosive GERD. The gastroesophageal reflux disease (GERD) can be, for example, symptomatic gastroesophageal reflux disease, erosive gastroesophageal reflux disease, pregnancy-induced gastroesophageal reflux disease, and the like. In other embodiments, the invention provides methods for treating and preventing gastroesophageal reflux disease in infants and children. Symptomatic GERD is characterized by the presence of symptoms of GERD, most commonly heartburn, which are related to the reflux of gastric contents into the esophagus. Symptomatic GERD is distinguished from GERD (or erosive GERD) by the absence of erosions in the esophageal mucosa.

In other embodiments, the invention provides methods for providing on-demand relief of GERD and/or symptoms associated with gastroesophageal reflux disease (GERD) by administering to a patient a therapeutically effective amount of at least one aminobenzoic acid compound. On-demand relief is accomplished by administering the aminobenzoic acid compound prior to consumption of foods/beverages that are known by the patient to cause GERD; during consumption of foods/beverages that are known by the patient to cause GERD; immediately following consumption of foods/beverages that are known by the patient to cause GERD; and/or after symptoms of GERD to begin to occur. On-demand relief does not require daily dosing of aminobenzoic acid compounds, but taking the medication to prevent the occurrence of GERD or taking the medication to treat the symptoms of GERD on an as-need basis. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing diarrhea by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Exemplary types of diarrhea include toddler's diarrhea, traveler's diarrhea, infectious diarrhea, chronic diarrhea, antibiotic-induced diarrhea, food allergy-induced diarrhea, diarrhea associated with carinoid tumors, and the like. The diarrhea can be diarrhea in immunocompromised patients, such as chronic diarrhea in immunocompromised patients. The food allergy-induced diarrhea can include, for example, diarrhea caused by lactose intolerance.

The invention provides methods for treating and preventing inflammatory bowel disease by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Inflammatory bowel disease refers to chronic inflammatory disorders involving the gastrointestinal tract, and is characterized by symptoms of diarrhea, bloody diarrhea, perianal sepsis, and/or abdominal pain. Inflammatory bowel disease includes ulcerative colitis, Crohn's disease, collagenous colitis, and lymphocytic colitis. Ulcerative colitis is an inflammatory reaction primarily involving the colonic mucosa, and is characterized by symptoms of bloody diarrhea, abdominal pain, fever, and/or weight loss. Crohn's disease is a chronic inflammation extending through all layers of the intestinal wall and involving the mesentery and regional lymph nodes and can involve the small bowel and/or colon. Crohn's disease is characterized by symptoms of fever, abdominal pain, diarrhea often without blood, fatigue, and/or weight loss. Collagenous colitis and lymphocytic colitis are characterized by signs of mucosal inflammation and symptoms of chronic watery diarrhea.

The invention provides methods for treating and preventing infectious enteritis by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Infectious enteritis refers to pathogen-induced diarrhea. Infectious enteritis can be caused by an infection from, for example, Campylobacter species, Shigella species, Yersinia species, such as Yersinia enterocolitica, Cryptosporidium species, Giardia species, such as Giardia lamblia, Salmonella species, Pseudomonas species, such as Pseudomonas aeruginosa, and the like.

The invention provides methods for treating and preventing idiopathic gastric acid hypersecretion by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastritis by administering to a patient in need thereof a therapeutically effective amount of at least one aminobenzoic acid compound. “Gastritis” refers to inflammation of the gastric mucosa. The term “gastritis” includes acute gastritis and chronic gastritis. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing constipation by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The constipation can be, for example, functional constipation, constipation induced by narcotics, constipation induced by diabetes, constipation induced by Parkinson' disease, constipation associated with multiple sclerosis, constipation caused by paraplegia, constipation caused by cerebral palsy, constipation caused by spina bifida, constipation caused by muscular dystrophy, constipation caused by Schogren's syndrome, constipation caused by cholinergic drugs, and the like.

The invention provides methods for treating and preventing colic by administering to a patient a therapeutically effective amount of at least one of the aminobenzoic acid compounds described herein. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The colic can be, for example, equine colic, pediatric colic, and the like.

The invention provides methods for treating and preventing vomiting by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The vomiting can be, for example, cyclic vomiting, cyclic vomiting in children, chemotherapy induced vomiting, radiation-induced vomiting, hyper emesis gravidirin, and the like.

The invention provides methods for treating and preventing nausea by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The nausea can be, for example, antibiotic-induced nausea, chemotherapy induced nausea, radiation-induced nausea, and the like.

The invention provides methods for treating and preventing motion sickness by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating gastrointestinal injuries, esophageal injuries, and/or gastric mucosal injuries in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing malabsorption syndromes by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. In one embodiment, the malabsorption syndrome is Whipple's disease. Whipple's disease is a malabsorption disease that interferes with the body's ability to absorb certain nutrients. Whipple's disease can cause weight loss, irregular breakdown of carbohydrates and fats, resistance to insulin, and malfunctions of the immune system. Symptoms of Whipple's disease include diarrhea, intestinal bleeding, abdominal bloating and cramps, loss of appetite, weight loss, fatigue, and weakness. In another embodiment, the malabsorption syndrome is Celiac disease. Celiac disease is a digestive disease that damages the small intestine and interferes with absorption of nutrients from food.

The invention provides methods for treating and preventing short bowel syndrome by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Treating short bowel syndrome includes treating the bleeding associated with or caused by short bowel syndrome.

The invention provides methods for treating and preventing bowel dysfunctions by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The bowel dysfunction can be an acute bowel dysfunction, a chronic bowel dysfunction, a post-surgical bowel dysfunction, and the like.

The invention provides methods for treating and preventing early satiety by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing abdominal pain by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing abdominal bloating by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing sour stomach by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing radiation-induced injury to the gastrointestinal tract by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastrointestinal disorders induced by NSAIDs in a patient in need thereof by administering at least one aminobenzoic acid compound. The aminobenzoic acid compound can optionally be administered in combination with an NSAID or can be administered in the form of a composition with an NSAID. In preferred embodiments, the invention provides methods for treating and preventing gastrointestinal bleeding and/or ulcers (e.g., peptic ulcers) induced by NSAIDs. Administering one or more of the aminobenzoic acid compounds of the invention can heal gastrointestinal ulcers caused by NSAIDs. All NSAIDs have the potential to cause damage to the gastrointestinal tract, and have been associated with inducing peptic ulcers (e.g., gastric and duodenal ulcers) and gastrointestinal bleeding. NSAIDs cause gastrointestinal damage by two mechanisms: (1) a topical effect that is pH and pKa related, and (2) a systemic effect mediated by cyclooxygenase (COX) inhibition with a reduction in prostaglandin synthesis. The NSAIDs may be COX-1 inhibitors and/or COX-2 inhibitors and/or COX-3 inhibitors. Any NSAID can be used in the compositions and methods of the invention. Exemplary COX-2 inhibitors include celecoxib, rofecoxib, valdecoxib, and the like. Exemplary NSAIDs include celecoxib, rofecoxib, valdecoxib, ibuprofen, acetaminophen, acetaminophen/aspirin/caffeine, aspirin, naproxen, ketorolac, ketoprofen, diflunisal, salsalate, salicylate, salicylamide, thiosalicylate, trisalicylate, mesalamine, sulfasalazine, methylsalicylate, phenylbutazone, oxyphenbutazone, antipyrine, aminopyrine, dipyrone, azapropazone, phenacetin, indomethacin, sulindac, mefenamic, meclofenamic, flufenamic, tolfenamic, etofenamic, tolmetin, naproxen, flurbiprofen, fenoprofen, fenbufen, pirprofen, oxaprozin, indoprofen, tiaprofenic acid, piroxicam, ampiroxicam, tenoxicam, tolmetin, meloxicam, tenidap, diclofenac, diclofenac/misoprostol, sulindac, etodolac, nabumentone, and the like. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing gastrointestinal disorders induced by steroids in a patient in need thereof by administering at least one aminobenzoic acid compound. The aminobenzoic acid compound can optionally be administered in combination with a steroid or can be administered in the form of a composition with a steroid. In preferred embodiments, the invention provides methods for treating and preventing gastrointestinal bleeding and/or ulcers (e.g., peptic ulcers) induced by steroids. Any steroid can be used in the compositions and methods of the invention. Exemplary steroids include alclometasone, beclomethasone, betamethasone, budesonide, clobetasol, clotrimazole/betamethasone, desonide, diflorasone, fluocinolone, fluocinolone, flurandrenolide, fluticasone, prednisone, methylprednisolone, hydrocortisone, flunisolide, hydrocortisone/pramoxine, triamcinolone, butixocort, dexamethasone, desoximetasone, halobetasol, fluocortin, tixocortal, tipredane, mometasone, prednicarbate, prednisone, and the like. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating or preventing gastrointestinal disorders induced by cholinergic compounds in a patient in need thereof by administering at least one aminobenzoic acid compound. The aminobenzoic acid compound can optionally be administered in combination with a cholinergic compound or can be administered in the form of a composition with a cholinergic compound. In preferred embodiments, the invention provides methods for treating and preventing gastrointestinal bleeding and/or ulcers (e.g., peptic ulcers) induced by cholinergic compounds. Exemplary cholinergic compounds include bethanecol, metoclopramide and the like. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing chronic sore throat by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing noncardiac chest pains by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing coughing by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for increasing gastric mucin production in a patient in need thereof by administering a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing dysphagia by administering to a patient a therapeutically effective amount of at least one of the aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like.

The invention provides methods for treating and preventing Shwachman syndrome by administering to a patient a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The primary features of the syndrome can include neutropenia, which can lead to reduced ability to fight general illness and mouth ulcers, gum disease, tooth decay, and/or ear infections. Other primary features can include an inability to produce enough enzymes to digest food sufficiently, bone abnormalities and growth retardation. Some children also have developmental delay.

The invention provides methods for treating and preventing one or more symptoms associated with iron deficiency anemia by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The following are the most common symptoms of iron-deficiency anemia: abnormal paleness or lack of color of the skin, irritability, fatigue (e.g., lack of energy or tiring easily), tachycardia, sore or swollen tongue, enlarged spleen, and/or a desire to eat peculiar substances, such as dirt or ice (e.g., pica).

The invention provides methods for decreasing nasal airway resistance and increasing nasal air flow by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. In other embodiments, the invention provides methods for decreasing nasal airway resistance during exercise by administering to a patient in need thereof a therapeutically effective amount of at least one aminobenzoic acid compound.

The invention provides methods for treating and preventing pancreatitis by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. Pancreatitis is an inflammation of the pancreas which can be acute or chronic. Symptoms of pancreatitis can include a swollen and tender abdomen, nausea, vomiting, fever and a rapid pulse. Pancreatitis can be acute or chronic.

The invention provides methods for treating and preventing cystic fibrosis by administering to a patient in need thereof a therapeutically effective amount of at least one aminobenzoic acid compound. The methods can optionally further comprise administering one or more proton pump inhibitors, histamine antagonists, antacids, bismuth compounds, VIP (or analogs or fragments thereof), secretin (or analogs or fragments thereof), sucralfate, cisapride, misoprostol, vitamin supplements, and the like. The patient can be an adult or a child. Cystic fibrosis is a disorder of the cells that line the lungs, small intestines, sweat glands and pancreas, where mucus contributes to the destruction of lung tissue. Symptoms of cystic fibrosis include excessive appetite, poor weight gain, diarrhea, persistent cough, and other digestive disorders.

The invention provides methods for treating and/or preventing psychiatric disorders by administering to a patient in need thereof a therapeutically effective amount of at least one aminobenzoic acid compound. In other embodiments, the invention provides methods for treating and/or preventing psychiatric disorders by administering to a patient in need thereof a therapeutically effective amount of at least one aminobenzoic acid compound and at least one cholinesterase inhibitor, and, optionally, at least one psychiatric medication. In other embodiments, the invention provides methods for treating and/or preventing psychiatric disorders by administering to a patient in need thereof a therapeutically effective amount of at least one aminobenzoic acid compound and at least one psychiatric medication, and, optionally, at least one cholinesterase inhibitor.

Any cholinesterase inhibitor known in the art can be used depending on the psychiatric illness that is being treated. Exemplary cholinesterase inhibitors include donepezil, phenserine, tolserine, phenethylnorcymserine, ganstigmine, epastigmine, tacrine, physostigmine, pyridostigmine, neostigmine, rivastigmine, galantamine, citicoline, velnacrine, huperzine (e.g., huperzine A), metrifonate, heptastigmine, edrophonium, TAK-147 (i.e., 3-[1-(phenylmethyl)-4-piperidinyl]-1-(2,3,4,5-tetrahydro-1H-1-benzazepin-8-yl)-1-propanone fumarate or other salts thereof), T-82, upreazine, and the like.

Any psychiatric medicine in the art can be used depending on the psychiatric illness that is being treated. Psychiatric medicines include, for example, antidepressant; anti-psychotic medications (e.g., pimozide, chlorpromazine, phenothiazines, butyrophenone, thioxanthines, haloperidol, sulpride, clozapine, sulpiride, tiapride, bifemelane, amisulpride, risperidone, olanzapine, quetiapine, polycarbophil, ziprasidone, aripiprazole, iloperidone, trifluoperazine, loxapine, molindone, fluphenazine, thiothixene, perphenazine, prochlorperizine, perphenazine/amitryptiline, mesoridazine, thioridazine); mood stabilizers (e.g., lithium, divalproex, gabapentin, carbamazepine, lamotrigine, topiramate); anti-anxiety medications (e.g., hydroxyzine, doxepin, venlafaxine, paroxetine, meprobamate, NGD 91-3, and benzodiazepines such as alprazolam, flurazepam, oxazepam, triazolam, estazolam, chlordiazepoxide, lorazepam, quazepam, diazepam, tamazepam, clonazepam); stimulants (e.g., methylphenidate, dextroamphetamine, pemoline, dextroamphetamine/levoamphetamine), and the like. Antidepressants include, for example, tricyclic antidepressants (e.g., amitriptyline, desipramine, imipramine, nortirptyline); serotonin-specific reuptake inhibitors (e.g., fluoxetine, paroxetine, sertraline, citalopram, fluvoxamine); monoamine oxidase inhibitors (e.g., phenelzine, tranylcypromine, isocarboxazid); other antidepressants (e.g., venlafaxine, nefazodone, bupropion, mirtazapine, trazodone, thioridazine, protriptyline), and the like.

The psychiatric disorder can be any known in the art. Exemplary psychiatric disorders include obsessive-compulsive disorder, post-traumatic stress disorder, anxiety, panic attacks, schizophrenia, schizoaffective disorders, depression, mania, manic-depression (bipolar disorder), apathy, delirium, phobias, amnesia, eating disorders (e.g., bulimia, anorexia), and the like. In one embodiment, the psychiatric disorders include obsessive-compulsive disorder, post-traumatic stress disorder, panic attacks, schizophrenia, schizoaffective disorders, depression, mania, manic-depression (bipolar disorder), apathy, delirium, phobias, amnesia, and eating disorders (e.g., bulimia, anorexia). In another embodiment, the psychiatric disorders include obsessive-compulsive disorder, schizophrenia, schizoaffective disorders, depression, mania, manic-depression (bipolar disorder), apathy, delirium, and phobias. In another embodiment, the psychiatric disorders include obsessive-compulsive disorder, schizophrenia, schizoaffective disorders, depression, mania, and manic-depression (bipolar disorder).

The invention provides methods for treating and preventing schizophrenia and/or schizoaffective disorders by administering to a patient a therapeutically effective amount of at least one aminobenzoic acid compound. The methods can optionally further comprise administering at least one psychiatric medication and/or at least one cholinesterase inhibitor. “Schizophrenia” is a psychosis characterized by a disorder in the thinking processes, such as delusions and hallucinations, and extensive withdrawal of the patient's interest from other people and the outside world, and the investment of it in their own. As used herein, the term “schizophrenia” refers to reactive and process schizophrenias, including, for example, chronic schizophrenia, ambulatory schizophrenia, catatonic schizophrenia, disorganized schizophrenia, latent schizophrenia, paranoid schizophrenia, pseudoneurotic schizophrenia, residual schizophrenia, and simple schizophrenia.

The invention provides methods for treating and preventing delirium in a patient in need thereof by administering a therapeutically effective amount of at least one aminobenzoic acid compound. The methods can optionally further comprise administering at least one psychiatric medication and/or at least one cholinesterase inhibitor. “Delirium” refers to a clouded state of consciousness and confusion that is marked by difficulty in sustaining attention to stimuli, disordered thinking, defective perceptions, illusions, hallucinations, disordered sleep-wakefulness cycles, and/or motor disturbances. There are various categories of delirium covered by the invention, including, for example, post-operative delirium (where the onset of the delirium is after an operation) anxious delirium (in which the predominating symptom is an incoherent apprehension or anxiety); collapse delirium (caused by extreme physical depression induced by a shock, profuse hemorrhage, exhausting labor, and the like); low delirium (in which there is little excitement, either mental or motor, where ideas are confused and incoherent but follow each other slowly); muttering delirium (common in low fevers in which the patient is unconscious but constantly muttering incoherently); posttraumatic delirium (a posttraumatic neuropsychological disorder of the brain with disturbed consciousness, agitation, hallucinations, delusions and/or disorientation); toxic delirium (caused by a poison); and tremens delirium (a form of acute organic brain syndrome due to alcoholic withdrawal and marked by sweating, tremor, atonic dyspepsia, restlessness, anxiety, precordial distress, mental confusion, and hallucinations).

The invention provides methods of treating and preventing mania in a patient in need thereof by administering a therapeutically effective amount of at least one aminobenzoic acid compound. The methods can optionally further comprise administering at least one psychiatric medication and/or at least one cholinesterase inhibitor. “Mania” is an emotional disorder characterized by symptoms of euphoria, increased psychomotor activity, rapid speech, flight of ideas, decreased need for sleep, distractibility, irritability, increased sexual desire, increased energy, grandiosity, and/or poor judgment. “Hypomania” refers to a mild form of mania. Mania and hypomania often occur in bipolar disorder.

The invention provides methods for treating bipolar disorder in a patient in need thereof by administering to a patient a therapeutically effective amount of at least one aminobenzoic acid compound. The methods can optionally further comprise administering at least one psychiatric medication and/or at least one cholinesterase inhibitor. Bipolar disorder, also called manic-depressive illness, is characterized by cycling mood changes from highs (e.g., mania) to lows (e.g., major depression or dysthymia).

The invention provides methods for treating depression in a patient in need thereof by administering to a patient a therapeutically effective amount of at least one aminobenzoic acid compound. The methods can optionally further comprise administering at least one psychiatric medication and/or at least one cholinesterase inhibitor. “Depression” refers to and includes major depression and dysthymia. Major depression is characterized by a persistent sad, anxious and/or empty mood; feelings of hopelessness, pessimism, guilt, worthlessness, and/or helplessness; a loss of interest or pleasure in hobbies and activities, including sex; decreased energy or fatigue; difficulty concentrating, remembering and/or making decisions; insomnia, early-morning awakening or oversleeping; increased or decreased appetite; thoughts of suicide or death; suicide attempts; restlessness and/or irritability; and/or persistent physical symptoms that do not respond to treatment, such as headaches, digestive disorders and/or chronic pain. Major depression can be characterized by a few or many symptoms which can vary over time. Dysthymia refers to a less severe (sometimes chronic) form of major depression.

The invention provides methods for treating and preventing post-traumatic stress disorders by administering to a patient in need thereof at least one aminobenzoic acid compound. The methods can optionally further comprise administering at least one psychiatric medication and/or at least one cholinesterase inhibitor. “Post-traumatic stress disorder” is a psychiatric illness that can occur following a traumatic event in which there is the threat of injury or death to the patient or someone else. Symptoms can include recurrent distressing memories of the event, recurrent dreams of the event, flashback episodes, bodily reactions to situations that remind the person of the traumatic event, the inability to remember important aspects of the trauma, lack of interest in normal activities, feelings of detachment, reduced expression of moods, irritability or outburst of anger, sleeping difficulties, difficulty concentrating, hypervigilance, paleness, heart palpitations, headache, fever, fainting, dizziness, and agitation. Some possible complications can include depression, anxiety, unusual phobia to things that are not usually frightening to other people, alcohol abuse and/or drug abuse.

The invention provides methods for treating and preventing anxiety disorders or panic attacks by administering to a patient in need thereof at least one aminobenzoic acid compound. The methods can optionally further comprise administering at least one psychiatric medication and/or at least one cholinesterase inhibitor. “Panic attacks” refer to unexpected and repeated episodes of intense fear accompanied by physical symptoms that can include chest pain, heart palpitations, shortness of breath, dizziness or abdominal distress. Other symptoms can include terror, nausea, tingling or numbness in the hands, flushes or chills, sense of unreality, fear of losing control, going “crazy,” or doing something embarrassing, and fear of dying.

The invention provides methods for treating and preventing obsessive-compulsive disorder by administering to a patient in need thereof at least one aminobenzoic acid compound. The methods can optionally further comprise administering at least one psychiatric medication and/or at least one cholinesterase inhibitor. “Obsessive-compulsive disorder” refers to an anxiety disorder characterized by the presence of obsessions or compulsions. An “obsession” refers to a recurrent or persistent thought that is intrusive or inappropriate. A “compulsion” is a repetitive behavior a patient feels driven to perform such as a physical action (i.e., handwashing) or a mental action (i.e., praying, repeating words, counting). Symptoms of the disorder include obsessions or compulsions that cause significant distress or interference with every day life, and are not due to medical illness or drug use.

The invention provides methods for treating and preventing amnesia by administering to a patient in need thereof at least one aminobenzoic acid compound. The methods can optionally further comprise administering at least one psychiatric medication and/or at least one cholinesterase inhibitor. “Amnesia” refers to a disturbance in memory manifested by total or partial inability to recall past experiences. Symptoms of amnesia can include memory gaps, confusion, changes in emotion, difficulty in remembering recent events and/or events in the past, and disorientation. Cognitive impairments associated with amnesia can include difficulty thinking/concentrating, drops in IQ, and problems with fine/gross motor coordination.

The invention provides methods for treating apathy in a patient in need thereof by administering to a patient a therapeutically effective amount of at least one aminobenzoic acid compound. The methods can optionally further comprise administering at least one psychiatric medication and/or at least one cholinesterase inhibitor. “Apathy” refers to a slowing of cognitive processes and/or a lack of motivation as manifested by one or more of the following: lack of productivity, lack of initiative, lack of perseverance, diminished socialization or recreation, lack of interest in learning new things, lack of interest in new experiences, lack of emotional responsivity to positive or negative events, unchanging or flat affect, and/or absence of excitement or emotional intensity.

The invention describes novel methods for treating and preventing eating disorders by administering to a patient a therapeutically effective amount of at least one of the aminobenzoic acid compounds described herein. The eating disorder can be, for example, anorexia or bulimia. The methods can optionally further comprise administering at least one psychiatric medication and/or at least one cholinesterase inhibitor.

The invention provides methods for treating learning disabilities by administering at least one aminobenzoic acid compound to a patient in need thereof. The learning disability can be any known in the art. Exemplary learning disabilities include, for example, attention deficit hyperactivity disorder, autism, dyslexia, mental retardation, developmental delays, Shwachman syndrome and the like.

The invention provides methods for treating and preventing attention deficit hyperactivity disorder in a patient in need thereof by administering a therapeutically effective amount of at least one aminobenzoic acid compound. “Attention deficit hyperactivity disorder” (ADHD or ADD) is a neurological condition where the patient, including adults and children, has a reduced ability to maintain attention without distraction, has a reduced ability to control doing or saying something due to impulsivity, has a lack of appropriate forethought, and/or is restless.

The invention provides methods for treating and preventing autism in a patient in need thereof by administering a therapeutically effective amount of at least one aminobenzoic acid compound. “Autism” is a complex developmental disability that affects the functioning of the brain, and typically appears in a patient by the age of three. Autism impacts the normal development of the brain in the areas of social interaction and communication skills. Patients with autism typically have difficulties in verbal and non-verbal communication, social interactions, and/or leisure or play activities. The disorder makes it hard for them to communicate with others and relate to the outside world. In some cases, aggressive and/or self-injurious behavior may be present. Patients with autism may experience sensitivities in the senses, exhibit repeated body movements (e.g., hand flapping, rocking), have unusual responses to people or attachments to objects and/or resistance to changes in routines.

The invention provides methods for treating and preventing dyslexia in a patient in need thereof by administering a therapeutically effective amount of at least one aminobenzoic acid compound. “Dyslexia” is characterized by one or more of the following: a memory instability for letters, words, or numbers; tendency to skip over or scramble letters, words, and sentences; poor reading ability; poor concentration; distractibility; photophobia; tunnel; vision, delayed visual and phonetic processing; poor handwriting prone to size, spacing, and letter-sequencing errors; memory instability for spelling, grammar, math, names, dates, and lists; speech disorders such as slurring, stuttering, minor articulation errors, poor word recall, and auditory-input and motor-output speech lags; impaired concentration, distractibility, hyperactivity, or overactivity; difficulties with balance and coordination functions; headaches, nausea, dizziness, vomiting, motion sickness, abdominal complaints, excessive sweating, and bed-wetting; and/or poor self-esteem.

The invention provides methods for treating and preventing mental retardation by administering to a patient in need thereof a therapeutically effective amount of at least one aminobenzoic acid compound. “Mental retardation” refers to the below-average general intellectual function with associated deficits in adaptive behavior that occurs before age 18. Symptoms of mental retardation can include failure to meet intellectual developmental markers, persistence of infantile behavior, lack of curiosity, decrease learning ability, and inability to meet educational demands of school.

The invention provides methods for treating and preventing developmental delay by administering to a patient in need thereof a therapeutically effective amount of at least one aminobenzoic acid compound. “Developmental delay” refers to a child who fails to achieve certain skills as quickly as expected, i.e., a child not reaching developmental bench marks at the usual age. Signs of developmental delay can include the delay in walking and other motor skills, the inability to walk, language delay or inability to learn, abnormalities of vision or hearing, behavioral problems, and seizures.

The invention provides methods for treating and preventing sleep disorders by administering to a patient in need thereof at least one aminobenzoic acid compound. “Sleep disorders,” refer to a disruptive pattern of sleep that can include difficulty falling or staying asleep, falling asleep at inappropriate times, excessive total sleep time, or abnormal behaviors associated with sleep. Sleep disorders include, for example, sleep apnea, REM (rapid eye movement) disorders, sleep onset with depression, age-related sleep disorders, narcolepsy, sleep deprivation, and REM-deprived sleep disorders. Symptoms of sleep disorders can include awakening in the night, difficulty falling asleep, excessive daytime drowsiness, loud snoring, episodes of stopped breathing, sleep attacks during the day, daytime fatigue, depressed mood, anxiety, difficulty concentrating, apathy, irritability, loss of memory or decreased memory, and lower leg movements during sleep. “REM sleep” refers to the occasional periods of active dreaming during sleep. “Age related sleep disorders” refer to the increased difficulty of falling asleep, the increase of awakenings, the less time spent in deep dreamless sleep, which can lead to confusion and other metal changes. “Narcolepsy” refers to a sleep disorder associated with uncontrollable sleepiness and frequent daytime sleeping.

The invention provides methods for enhancing REM sleep by administering to a patient in need thereof at least one aminobenzoic acid compound. Enhancing REM sleep includes, for example, increasing the number of REM sleep episodes and/or increasing the duration of REM sleep episodes. Without intending to be bound by any theory of the invention, enhancing REM sleep may enhance memory consolidation and learning, and may improve a patient's mood.

The invention provides methods for treating and preventing one or more symptoms associated with or caused by sleep apnea by administering to a patient in need thereof a therapeutically effective amount of at least one aminobenzoic acid compound. Patients with sleep apnea stop breathing repeatedly during their sleep, often for a minute or longer, and as many as hundreds of times during a single night. In one embodiment, the sleep apnea is obstructive sleep apnea syndrome or obstructive sleep apnea, which is caused by a complete and/or partial obstruction of the patient's airway. Partial obstructive sleep apnea can also be called obstructive hypopnea, where hypopnea is slow, shallow breathing. Physical signs that suggest obstructive sleep apnea syndrome or obstructive sleep apnea include loud snoring, witnessed apneic episodes, obesity, excessive daytime sleepiness, and nocturnal snorting and gasping.

The invention provides methods for treating and preventing Tourette's syndrome in a patient in need thereof by administering a therapeutically effective amount of at least one aminobenzoic acid compound. “Tourette's syndrome” is characterized by motor incoordination, echolalia (i.e., repetition of what is said by other people) and/or coprolalia (i.e., involuntary utterances of vulgar or obscene words). Tourette's syndrome is a form of tic.

The invention provides methods for treating and preventing obesity by administering to a patient in need thereof a therapeutically effective amount of at least one aminobenzoic acid compound. In still other embodiments, the invention provides methods for treating patients who have had surgery for obesity (e.g., laparoscopic obesity surgery, bariatric surgery, gastric surgery, gastric bypass surgery) by administering a therapeutically effective amount of at least one aminobenzoic acid compound.

The invention provides methods for treating and preventing epilepsy or other seizure disorders by administering to a patient in need thereof a therapeutically effective amount of at least one aminobenzoic acid compound. Epilepsy, sometimes called a seizure disorder, is a chronic medical condition produced by temporary changes in the electrical function of the brain, causing seizures which affect awareness, movement and/or sensation.

The invention provides methods for treating and preventing post-menopausal symptoms (e.g., hot flashes and sweats) by administering to a patient a therapeutically effective amount of at least one aminobenzoic acid compound.

The invention provides methods for treating and preventing premenstrual syndrome by administering to a patient a therapeutically effective amount of at least one of the aminobenzoic acid compound.

The invention provides methods for treating and preventing asthma by administering to a patient a therapeutically effective amount of at least one aminobenzoic acid compound. The method can further comprise administering at lest one anti-asthma medication.

The invention provides methods for treating and preventing laryngitis by administering to a patient a therapeutically effective amount of at least one aminobenzoic acid compound.

The invention provides methods for treating and preventing migraines by administering to a patient a therapeutically effective amount of at least one aminobenzoic acid compound. In another embodiment, the invention provides methods for preventing and treating migraines by administering a therapeutically effective amount of at least one aminobenzoic acid compound and at least one cholinesterase inhibitor. In another embodiment, the invention provides methods for preventing and treating migraines by administering a therapeutically effective amount of at least one aminobenzoic acid compound and at least one migraine drug. In another embodiment, the invention provides methods for preventing and treating migraines by administering a therapeutically effective amount of at least one aminobenzoic acid compound, at least one cholinesterase inhibitor and at least one migraine drug. The aminobenzoic acid compound, the cholinesterase inhibitor and migraine drug can be administered separately or in the form of a composition.

The migraines can be classic migraines, common migraines, complicated migraines, and/or cluster headaches. In other embodiments, the migraines can be menstrual migraines, premenstrual migraines, ophthalmic migraines, and/or ophthalmoplegic migraines. In other embodiments, the migraines can be fulgurating migraines, Harris' migraines, and/or hemiplegic migraines. In still other embodiments, the migraines can be abdominal migraines.

“Migraine” refers to periodic, hemicranial, throbbing headaches that can be accompanied by nausea and/or vomiting. Migraines can occur in children and adults, and men and women. “Migraine” includes classic migraines, common migraines, complicated migraines, cluster headaches, menstrual migraines, premenstrual migraines, ophthalmic migraines, ophthalmoplegic migraines, fulgurating migraines, Harris' migraines, and/or hemiplegic migraines. Neurologic symptoms can occur which are caused by migraines, bur which are not followed by a headache. For example, abdominal pain and vomiting can occur without headache as the sole expression of a migraine. “Classic migraines” generally begin with neurologic symptoms such as visual scintillations, dazzling zigzag lines, photophobia and spreading scotomas, or dizziness and tinnitus. Classic migraines can have premonitory symptoms such as feelings of elation, excessive energy, thirst, cravings for sweets, and/or drowsiness. At other times, classic migraines can have premonitory symptoms such as a slowing of mentation, a feeling of impending doom, and/or depression. At other times, there can be no premonitory symptoms. “Common migraines” generally have an unheralded onset of headache that can be accompanied by nausea and/or vomiting. Unlike the classic migraine, the common migraine generally does not have neurologic symptoms that occur prior to the onset of the headache. “Complicated migraines” refers to migraines accompanied by neurologic symptoms (e.g., such as those described for classic migraines) that can either precede or accompany the headache. In complicated migraines, numbness and tingling of the lips, face, hand, arm, and/or leg on side of the body can occur, sometimes in combination with aphasic disorder. The arm and/or leg can become weak or paralyzed on one side, mimicking a stroke. The numbness or weakness can spread from one part of the body to another slowly over a period of minutes. “Complicated migraines” include basilar migraines. In basilar migraines, the visual disorder and paresthesias are bilateral and can be accompanied by confusion, stupor, coma, aggressive outbursts, vertigo, diplopia, and/or dysarthria. Basilar migraines occur in 30% of children with migraines. “Cluster headaches” are also called paroxysmal nocturnal cephalalgia, migrainous neuralgia, histamine headache, and Horton's syndrome. Cluster headaches are characterized by constant, unilateral orbital pain, with onset usually within two or three hours after falling asleep. The pain can be intense and steady with lacrimation, blocked nostril, then rhinorrhea, and sometimes miosis, ptosis, flush, and edema of cheek. “Menstrual migraines” refer to migraine headaches that can generally occur from about two days prior to a woman's menstrual cycle until about three days after a woman's menstrual cycle. In another embodiment, menstrual migraines refer to migraine headaches that can generally occur from about two days prior to a woman's menstrual cycle and that generally end on the last day of the woman's menstrual cycle. Menstrual migraines can occur or re-occur at any time during the menstrual cycle. “Premenstrual migraines” are migraine headaches that can generally occur from about seven days prior to a woman's menstrual cycle to about three days prior to a woman's menstrual cycle. Premenstrual migraines can occur or re-occur at any time during the premenstrual cycle. “Ophthalmic migraines” are migraine headaches that are generally accompanied by a marked disturbance of vision. “Ophthalmoplegic migraines” are migraine headaches associated with paralysis of the eye muscles. “Fulgurating migraines” are migraine headaches characterized by an abrupt beginning and severity. “Harris' migraine” is also known as periodic migrainous neuralgia. “Hemiplegic migraines” are a form of migraine headache associated with transient hemiplegia. “Abdominal migraines” are characterized by paroxysmal abdominal pain without apparent cause. “Treating” refers to eliminating the migraine or alleviating the symptoms of the migraine (e.g., compared to the symptoms prior to administering one or more aminobenzoic acid compounds and, optionally, one or more migraine drugs). Treating encompasses alleviating the number of migraines, the intensity of the migraines and/or the duration of the migraines.

Migraine drugs that can be used to prevent and/or treat migraines include, for example, estrogen, serotonin antagonists, non-steroidal antiinflammatory drugs (NSAIDs) (e.g., COX-1 inhibitors and/or COX-2 inhibitors), calcium channel blockers, beta-andrenergic blockers, anticonvulsants, and antidepressants (e.g., tricylcic antidepressants, monoamine oxidase inhibitors, and selective serotonin reuptake inhibitors). Estrogen is generally used for preventing and/or treating menstrual migraines and premenstrual migraines.

Exemplary migraine drugs that can be used to prevent and/or treat migraines include celecoxib, valdecoxib, meloxicam, etodolac, rofecoxib, PNU-142633, vigabatrin, topiramate, montelukast (e.g., the sodium salt thereof), gabapentin, piroxicam (e.g., piroxicam betadex), valproate (e.g., the semisodium salt thereof), ketoprofen, diclofenac (e.g., the potassium salt), tiagabine, botulinum, nebivolol, lisinopril, nimodipine, tizanidine, zolmitriptan, sumatriptan (e.g., the succinate salt thereof), rizatriptan (e.g., the benzoate salt thereof), pizotifen, oxetorone, naratriptan, lomerizine (e.g., the hydrochloride salt thereof), gepefrine, flunarizine, almotriptan, alpiropride, tolfenamic acid, migpriv, timolol (e.g., the maleate salt thereof), buclizine (e.g., the hydrochloride salt thereof), baclofen, methysergide (e.g., the maleate salt thereof), flunarizine (e.g., the hydrochloride salt thereof), cyproheptadine (e.g., the hydrochloride salt thereof), ergotamine (e.g., the tartrate salt thereof), lidocaine (e.g., the hydrochloride salt thereof), indoramin (e.g., the hydrochloride salt thereof), butorphanol, KT 2962, BMS 181885, ADDS-ergotamine, NPS-1776, GW-468816, triptan, Pharmaprojects No. 6313, MT-500, donitriptan (e.g., the mesylate salt thereof), ALX-0646, civamide, propanolol, zucapsaicin, CNS 5161, vofopitant, lanepitant, dapitant, ganaxolone, LY-53857, sergolexole (e.g., the maleate salt thereof), sumatriptan, MT-400, fluoxetine, (S)-fluoxetine, dihydroergotamine (e.g., the mesylate salt thereof), tonabersat, IS-159, BIBN-4096, metoclopramide, naproxen, MT-100 (e.g., a combination of metoclopramide and naproxen), dotarizine, frovatriptan, eletriptan, aspirin, ibuprofen, acetaminophen, amitryptiline, doxepin, ergot preparations, caffeine, cafergot (e.g., a combination of caffeine and ergotamine), codeine, meperidine, promethazine, atropine, phenobarbital, nifedipine, verapamil, chlorpromazine, lithium, prednisone, propranolol, phenelzine, mefenamic acid, flufenamic acid, LY334370, indomethacin, dichloralphenazone, isometheptene, butalbital, ketorolac, clonazepam, atenolol, metoprolol, nadolol, imipramine, nortripyline, diltiazem, valproic acid, divalproex, cyproheptadine, or pharmaceutically acceptable salts thereof.

Any aminobenzoic acid compound known in the art can be used in the compositions and methods described herein. In one embodiment, the aminobenzoic acid compound is represented by the compounds of formula (I), (II), (III), (IV), (V), (VI), (VII), (VIII), (IX), (X), (XI) and (XII). The compounds can be in the form of pharmaceutically acceptable salts known in the art. In other embodiments, the compounds can be in the form of stereoisomers.

In one embodiment, the aminobenzoic acid compound of the invention is a compound of Formula (I): 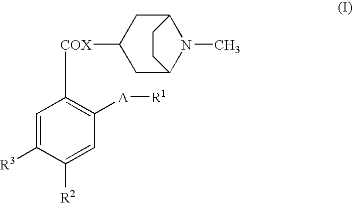
wherein R¹ is a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² is an amino, acylamino, carboxyamino or alkylamino group; R³ is a halogen atom; X is —O— or —NH—; and A is O or S. In other embodiments, the compound of formula (I) can be in the form of a pharmaceutically acceptable salt; in the form of a stereoisomer(s); or in the form of a pharmaceutically acceptable salt of a stereoisomer(s).

In one embodiment with respect to the compound of formula (I) there is a proviso that when R¹ is a saturated or unsaturated heterocyclic group, the saturated or unsaturated heterocyclic group is one exclusive of tetrahydrofuranyl and 1,3-benzodioxolanyl, that when R¹ represents a saturated or unsaturated heterocyclicalkyl group, the saturated or unsaturated heterocyclic group constituting the saturated or unsaturated heterocyclicalkyl group is one exclusive of tetrahydrofuranyl and 1,3-benzodioxolanyl, that when A represents an oxygen atom and R¹ represents a halogen-substituted lower alkyl group, the group represented by the formula: -A-R¹ is a group exclusive of one represented by the formula: —O—(CH²)ᵣ—CF₃ (wherein r is 0 or an integer of 1 to 4) and that when R¹ represents an unsubstituted cycloalkylalkyl group, the unsubstituted cycloalkylalkyl group is one exclusive of one represented by the formula: —(CH₂)ₘ—R (wherein R is a cycloalkyl group and m is an integer of 1 to 6).

In one embodiment, the aminobenzoic acid compound of the invention is a compound of Formula (II): 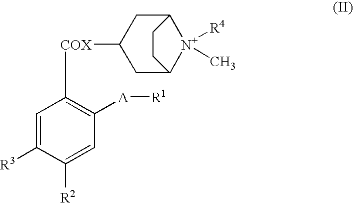
wherein R¹ is a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² is an amino, acylamino, carboxyamino or alkylamino group; R³ is a halogen atom; X is: —O— or —NH—; A is O or S; and R⁴ is a lower alkyl or aralkyl group. In other embodiments, the compound of formula (II) can be in the form of a pharmaceutically acceptable salt; in the form of a stereoisomer(s); or in the form of a pharmaceutically acceptable salt of a stereoisomer(s).

In one embodiment, the aminobenzoic acid compound of the invention is a compound of Formula (III): 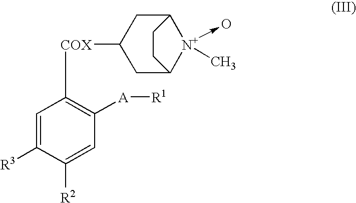
wherein R¹ represents a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² represents an amino, acylamino, carboxyamino or alkylamino group; R³ represents a halogen atom; X represents a group represented by the formulas: —O— or —NH—; and A represents an oxygen or sulfur atom. In other embodiments, the compound of formula (III) can be in the form of a pharmaceutically acceptable salt; in the form of a stereoisomer(s); or in the form of a pharmaceutically acceptable salt of a stereoisomer(s).

Specific examples of the aminobenzoic acid compounds according to the invention include: (1) compounds represented by Formulas (I), (II) and/or (III) with the proviso that R¹ is an alkynyl group; (2) compounds represented by the Formulas (I), (II) and/or (III) with the proviso that R² is an amino group; (3) compounds represented by the Formula (I), with the proviso that R¹ is a cyanoalkyl, cyanoalkenyl, substituted cycloalkyl, substituted cycloalkylalkyl, halogen-substituted lower alkyl, alkenyl, alkynyl, saturated or unsaturated heterocyclicalkyl or saturated or unsaturated heterocyclic group or a group represented by the formula: —CH(CH₃)R′ (wherein R′ is a cyclopentyl group); and (5) compounds represented by Formulas (II) and (III).

In one embodiment, the aminobenzoic acid compound of the invention is a compound of Formula (IV):

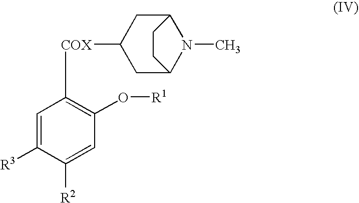

wherein R¹ₐ represents an alkynyl group; R² represents an amino, acylamino, carboxyamino or alkylamino group; R³ represents a halogen atom; and X represents a group represented by the formulas: —O— or —NH—. In other embodiments, the compound of formula (IV) can be in the form of a pharmaceutically acceptable salt; in the form of a stereoisomer(s); or in the form of a pharmaceutically acceptable salt of a stereoisomer(s).

In one embodiment, the aminobenzoic acid compound of the invention is a compound of Formula (V): 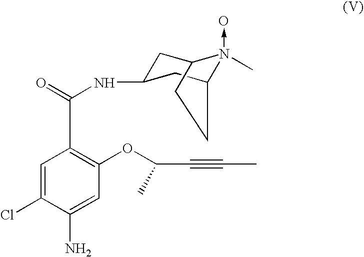
In other embodiments, the compound of formula (V) can be in the form of a pharmaceutically acceptable salt; in the form of a stereoisomer(s); or in the form of a pharmaceutically acceptable salt of a stereoisomer(s).

Other specific examples of the pharmaceutically acceptable salt of the aminobenzoic acid compound according to the invention include the compound represented by Formula (VI):

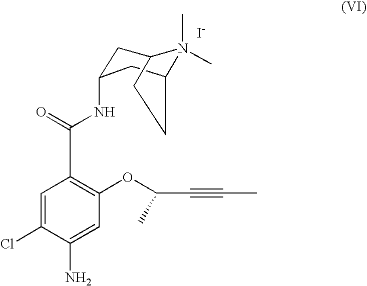

In the compounds of Formula (I)-(IV), the lower alkyl group constituting the halogen-substituted lower alkyl group as defined with respect to R¹ is a straight-chain or branched alkyl group having 1 to 8 carbon atoms and examples thereof include methyl, ethyl, propyl, isopropyl, butyl, isobutyl, sec-butyl, tert-butyl, pentyl(amyl), isopentyl, neopentyl, tert-pentyl, 1-methylbutyl, 2-methylbutyl, 3-methylbutyl, 1,2-dimethylpropyl, hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl and octyl groups. Further, the halogen atom constituting the halogen-substituted lower alkyl group includes fluorine, chlorine, bromine and iodine atoms and the number of the substituting halogen atoms is preferably 1 to 3. Furthermore, the halogen atom may be bonded to any of the carbon atoms constituting the lower alkyl group, with the proviso that when the aminobenzoic acid compound is selected from the compounds consisting of those represented by formula (I)-(IV), a case wherein the group represented by the formula: -A-R¹ in formula (I)-(IV) is a group represented by the formula: —O—(CH₂)ᵣ—CF₃ (wherein r is 0 or an integer of 1 to 4) is excepted.

The alkyl group constituting the cyanoalkyl group as defined with respect to R¹ is a lower alkyl group as described in the definition of the halogen-substituted lower alkyl group (hereinafter abbreviated to “the above-defined lower alkyl group”). The cyano group may be bonded to any of the carbon atoms constituting the alkyl group and the alkyl group is preferably substituted by 1 or 2 cyano groups.

The alkenyl group as defined with respect to R¹ is one derived from the above-defined lower alkyl group in which one or more carbon-carbon single bonds are replaced by carbon-carbon double bonds, though the double bonds may be present at arbitrary positions.

The alkynyl group as defined with respect to R¹ is one derived from the above-defined lower alkyl group in which one or more carbon-carbon single bonds are replaced by triple bonds, though the triple bonds may be present at arbitrary positions. Examples of the alkynyl group are as follows, though the alkynyl groups according to the present invention is not limited to them:

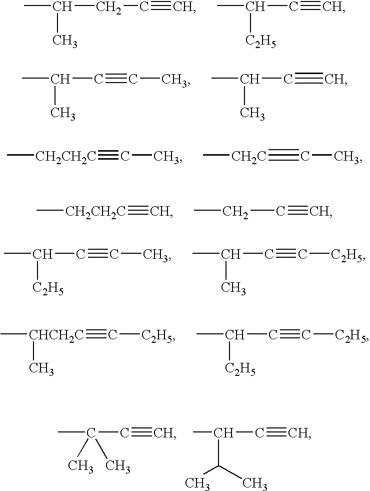

Other examples thereof include groups represented by the formulas:

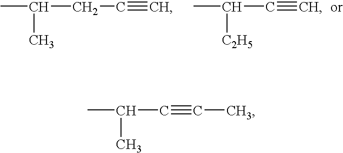

The cyanoalkenyl group as defined with respect to R¹ is an alkenyl group as defined above which is substituted by one or two cyano groups at arbitrary positions.

The cycloalkyl group constituting the substituted cycloalkyl group as defined with respect to R¹ is one having 3 to 8 carbon atoms, preferably 5 or 6 carbon atoms. Preferable examples of the substituent include lower alkyl, lower alkoxy, carboxyl, cyano and amino groups.

The cycloalkyl group constituting the substituted or unsubstituted cycloalkylalkyl group as defined with respect to R¹ is the cycloalkyl group as described above. Further, the substituent of the substituted cycloalkylalkyl group is a group as defined with respect to the substituent of the above-defined substituted cycloalkyl group.

The alkyl group constituting the substituted or unsubstituted cycloalkylalkyl group as defined with respect to R¹ is the above-defined lower alkyl group. The substituted or unsubstituted cycloalkyl group may be bonded to any of the carbon atoms constituting the alkyl group and the alkyl group is preferably substituted by one substituted or unsubstituted cycloalkyl group.

When R¹ represents an unsubstituted cycloalkylalkyl group, the alkyl group is preferably a branched alkyl group. Further, when the aminobenzoic acid compound is selected from the compounds consisting of those represented by formula (I)-(IV) and when R¹ represents an unsubstituted cycloalkylalkyl group, the unsubstituted cycloalkylalkyl group is one exclusive of one represented by the formula: —(CH₂)ₘ—R (wherein R is a cycloalkyl group and m is an integer of 1 to 6).

The saturated or unsaturated heterocyclic group as defined with respect to R¹ is a saturated or unsaturated 5- to 7-membered ring group having one or two oxygen, nitrogen and/or sulfur atoms. When the aminobenzoic acid compound is selected from the compounds consisting of those represented by formula (I)-(IV), the saturated or unsaturated heterocyclic group is a group exclusive of tetrahydrofuranyl and 1,3-benzodioxolanyl.

The saturated or unsaturated heterocyclic group constituting the saturated or unsaturated hetero-cyclicalkyl group as defined with respect to R¹ is a heterocyclic group as described above in the definition of the saturated or unsaturated heterocyclic group. When the aminobenzoic acid compound is selected from the compounds consisting of those represented by Formula (I)-(IV), the saturated or unsaturated heterocyclic group constituting the saturated or unsaturated heterocyclicalkyl group is a group exclusive of tetrahydrofuranyl and 1,3-benzodioxolanyl.

The alkyl group of the saturated or unsaturated heterocyclicalkyl group is the above-defined lower alkyl group. The saturated or unsaturated heterocyclic group may be bonded to any of the carbon atoms constituting the alkyl group and the alkyl group is preferably substituted by one saturated or unsaturated heterocyclic group.

The acyl group constituting the acylamino group as defined with respect to R² may be one derived from any carboxylic acid selected from among aliphatic saturated carboxylic acids, aliphatic unsaturated carboxylic acids, saturated and unsaturated carbocyclic carboxylic acids, heterocyclic carboxylic acids, hydroxy carboxylic acids, and alkoxy carboxylic acids, and examples of the acyl group include lower alkanoyl groups such as formyl, acetyl, propionyl, butyryl, valeryl, isovaleryl and pivaloyl groups; aroyl groups such as benzoyl, toluoyl and naphthoyl groups; and heteroaroyl groups such as furoyl, nicotinoyl and isonicotinoyl groups.

The acylamino group may be either a monosubstituted [—NH-(acyl group)] or disubstituted one

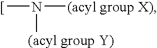

wherein the acyl groups X and Y may be same or different from each other], the former being preferable.

The carboxyamino group as defined with respect to R² is a group represented by the formula: —NH—COOH.

The alkyl group constituting the alkylamino group as defined with respect to R² is the above-defined lower alkyl group. The alkylamino group may be either a monosubstituted [—NH-(alkyl group)] or disubstituted one

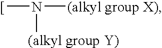

wherein the alkyl groups X and Y may be same or different from each other], the former being preferable.

The halogen atom as defined with respect to R³ includes fluorine, chlorine, bromine and iodine atoms, among which chlorine atom is preferable.

The lower alkyl group as defined with respect to R⁴ is the above-defined lower alkyl group as defined with respect to R¹.

The alkyl group constituting the aralkyl group as defined with respect to R⁴ is the above-defined lower alkyl group as defined with respect to R¹. The aralkyl group as defined with respect to R⁴ is one wherein a hydrogen atom of the lower alkyl group constituting the aralkyl group is substituted by an aryl group.

The lower alkynyl group having 3 to 10 carbon atoms as defined with respect to R⁵ is the alkynyl group as defined with respect to R¹, with the proviso that the number of carbon atoms is 3 to 10.

The lower alkynyl group having 3 to 10 carbon atoms defined with respect to R⁵ of the general formula (a) includes propargyl, 1-propyn-1-yl, 1-butyn-1-yl, 2-butyn-1-yl, 3-butyn-1-yl, 3-butyn-2-yl, 1-pentyn-1-yl, 1-pentyn-3-yl, 1-pentyn-4-yl, 2-pentyn-1-yl, 3-pentyn-1-yl, 3-pentyn-2-yl, 4-pentyn-1-yl, 3-methyl-1-butyn-1-yl, 2-methyl-3-butyn-1-yl, 3-methyl-1-butyn-3-yl, 1-hexyn-1-yl, 1-hexyn-3-yl, 1-hexyn-4-yl, 1-hexyn-5-yl, 2-hexyn-1-yl, 2-hexyn-4-yl, 2-hexyn-5-yl, 3-hexyn-1-yl, 3-hexyn-2-yl, 4-hexyn-1-yl, 5-hexyn-1-yl, 1-methyl-3-pentyn-2-yl, 3,3-dimethyl-1-butyn-1-yl, 2,2-dimethyl-3-butyn-1-yl, 4-heptyn-1-yl, 4-heptyn-2-yl and 4-heptyn-3-yl groups. When R.sup.4 has a substituent, the substituent is, for example, a lower alkyl, lower cycroalkyl, lower alkenyl, lower halogenated alkyl, lower alkoxy, cyano, lower cyanoalkyl, aryl or arylalkyl group or a halogen atom.

In another embodiment, the aminobenzoic acid compound of the invention is a compound of Formula (VII): 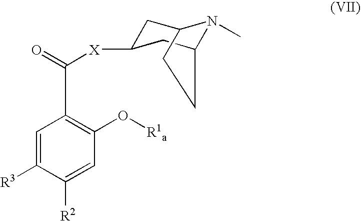
wherein R¹ₐ is an alkynyl group; and X, R² and R³ are each as defined above. In one embodiment, X is —NH—; R² is an amino or acylamino group; and R³ is chlorine. In other embodiments, the compound of formula (VII) is in the form of a pharmaceutically acceptable salt; in the form of a stereoisomer(s); or in the form of a pharmaceutically acceptable salt of a stereo isomer.

Other examples of the alkynyl group for R¹ₐ are:

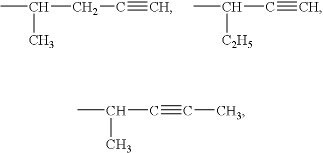

One preferred compound of the invention is (−)-endo-4-amino-5-chloro-N-(8-methyl-8-azabicyclo[3.2.1]oct-3-yl)-2-{(3-pentyn-2-yl)oxy}benzamide, a pharmaceutically acceptable salt thereof, and/or a stereoisomer thereof. Another preferred compound is (−)-endo-4-amino-5-chloro-N-(8-methyl-8-azabicyclo[3.2.1]oct-3-yl)-2-{(3-pentyn-2-yl)oxy}benzamide hydrochloride.

Other preferred compounds of the invention include endo-4-amino-5-chloro-N-(8-methyl-8-azabicyclo[3.2.1]oct-3-yl)-2-{(3-pentyn-2-yl)oxy}benzamide; endo-4-amino-5-chloro-N-(8-methyl-8-azabicyclo[3.2.1]oct-3-yl)-2-{(3-pentyn-2-yl)oxy}benzamide hydrochloride; (−)-endo-4-amino-5-chloro-N-(8-methyl-8-azabicyclo[3.2.1]oct-3-yl)-2-{(3-pentyn-2-yl)oxy}benzamide; (+)-endo-4-amino-5-chloro-N-(8-methyl-8-azabicyclo[3.2.1]oct-3-yl)-2-{(3-pentyn-2-yl)oxy}benzamide; (−)-endo-4-amino-5-chloro-N-(8-methyl-8-azabicyclo[3.2.1]oct-3-yl)-2-{(3-pentyn-2-yl)oxy)benzamide hydrochloride; endo-4-amino-5-chloro-N-(8-methyl-8-azabicyclo[3.2.1]oct-3-yl)-2-{(1-pentyn-3-yl)oxy}benzamide; endo-4-amino-5-chloro-N-(8-methyl-8-azabicyclo[3.2.1]oct-3-yl)-2-propargyloxybenzamide; endo-4-amino-2-(2-butynyloxy)-5-chloro-N-(8-methyl-8-azabicyclo[3.2.1]oct-3-yl)benzamide; endo-4-amino-5-chloro-N-(8-methyl-8-azabicyclo[3.2.1]oct-3-yl)-2-(3-pentynyloxy)benzamide; endo-4-amino-2-{(3-butyn-2-yl)oxy}-5-chloro-N-(8-methyl-8-azabicyclo[3.2.1]oct-3-yl)benzamide; endo-4-amino-5-chloro-2-{(4-heptyn-3-yl)oxy}-N-(8-methyl-8-azabicyclo[3.2.1]oct-3-yl)benzamide; endo-4-amino-5-chloro-2-{(4-heptyn-2-yl)oxy}-N-(8-methyl-8-azabicyclo[3.2.1]oct-3-yl)benzamide; endo-4-amino-5-chloro-2-{(2-hexyn-4-yl)oxy}-N-(8-methyl-8-azabicyclo[3.2.1]oct-3-yl)benzamide; endo-4-amino-5-chloro-2-{(3-hexyn-2-yl)oxy}-N-(8-methyl-8-azabicyclo[3.2.1]oct-3-yl)benzamide; endo-4-amino-5-chloro-2-{(1-hexyn-3-yl)oxy}-N-(8-methyl-8-azabicyclo[3.2.1]oct-3-yl)benzamide; endo-4-amino-5-chloro-2-(2-fluoroethoxy)-N-(8-methyl-8-azabicyclo[3.2.1]oct-3-yl)benzamide; endo-4-amino-5-chloro-2-(3-cyanopropoxy)-N-(8-methyl-8-azabicyclo[3.2.1]oct-3-yl)benzamide; endo-4-amino-5-chloro-2-(1-cyclopentylethoxy)-N-(8-methyl-8-azabicyclo[3.2.1]oct-3-yl)benzamide; endo-4-amino-5-chloro-N-(8-methyl-8-azabicyclo[3.2.1]-oct-3-yl)-2-(tetrahydro-4H-pyran-4-oxy)benzamide; endo-4-amino-5-chloro-2-(4-methoxycyclohexyloxy)-N-(8-methyl-8-azabicyclo[3.2.1]oct-3-yl)benzamide; endo-4-amino-5-chloro-N-(8-methyl-8-azabicyclo[3.2.1]oct-3-yl)-2-{(1-pentyn-3-yl)oxy}benzamide hydrochloride; endo-4-amino-5-chloro-(8-methyl-8-azabicyclo[3.2.1]oct-3-yl)-2-propargyloxy benzamide hydrochloride; endo-4-amino-2-(2-butynyloxy)-5-chloro-N-(8-methyl-8-azabicyclo[3.2.1]oct-3-yl)benzamide hydrochloride; endo-4-amino-5-chloro-N-(8-methyl-8-azabicyclo[3.2.1]oct-3-yl)-2-(3-pentynyloxy)benzamide hydrochloride; endo-4-amino-2-{(3-butyn-2-yl)oxy}-5-chloro-N-(8-methyl-8-azabicyclo[3.2.1]oct-3-yl)benzamide hydrochloride; endo-4-amino-5-chloro-2-{(4-heptyn-3-yl)oxy)-N-(8-methyl-8-azabicyclo[3.2.1]oct-3-yl)benzamide hydrochloride; endo-4-amino-5-chloro-2-{(4-heptyn-2-yl)oxy}-N-(8-methyl-8-azabicyclo[3.2.1]oct-3-yl)benzamide hydrochloride; endo-4-amino-5-chloro-2-{(2-hexyn-4-yl)oxy}-N-(8-methyl-8-azabicyclo[3.2.1]oct-3-yl)benzamide hydrochloride; endo-4-amino-5-chloro-2-{(3-hexyn-2-yl)oxy}-N-(8-methyl-8-azabicyclo[3.2.1]oct-3-yl)benzamide hydrochloride; endo-4-amino-5-chloro-2-{(1-hexyn-3-yl)oxy}-N-(8-methyl-8-azabicyclo[3.2.1]oct-3-yl)benzamide hydrochloride; endo-4-amino-5-chloro-2-(2-fluoroethoxy)-N-(8-methyl-8-azabicyclo[3.2.1]oct-3-yl)benzamide hydrochloride; endo-4-amino-5-chloro-2-(3-cyanopropoxy)-N-(8-methyl-8-azabicyclo[3.2.1]oct-3-yl)benzamide hydrochloride; endo-4-amino-5-chloro-2-(1-cyclopentylethoxy)-N-(8-methyl-8-azabicyclo[3.2.1]oct-3-yl)benzamide hydrochloride; endo-4-amino-5-chloro-N-(8-methyl-8-azabicyclo[3.2.1]-oct-3-yl)-2-(tetrahydro-4H-pyran-4-oxy)benzamide hydrochloride; endo-4-amino-5-chloro-2-(4-methoxycyclohexyloxy)-N-(8-methyl-8-azabicyclo[3.2.1]oct-3-yl)benzamide hydrochloride; (−)-endo-4-amino-5-chloro-N-(8-methyl-8-azabicyclo[3.2.1]oct-3-yl)-2-{(1-pentyn-3-yl)oxy}benzamide; (+)-endo-4-amino-5-chloro-N-(8-methyl-8-azabicyclo[3.2.1]oct-3-yl)-2-{(1-pentyn-3-yl)oxy}benzamide; (+)-endo-4-amino-5-chloro-N-(8-methyl-8-azabicyclo[3.2.1]oct-3-yl)-2-{(3-pentyn-2-yl)oxy}benzamide hydrochloride; (−)-endo-4-amino-5-chloro-N-(8-methyl-8-azabicyclo[3.2.1]oct-3-yl)-2-{(1-pentyn-3-yl)oxy}benzamide hydrochloride; (+)-endo-4-amino-5-chloro-N-(8-methyl-8-azabicyclo[3.2.1]oct-3-yl)-2-{(1-pentyn-3-yl)oxy}benzamide hydrochloride; endo-4-amino-5-chloro-N-(8-methyl-8-azabicyclo[3.2.1]oct-3-yl)-2-{(4-pentyn-2-yl)oxy}benzamide; endo-4-amino-5-chloro-N-(8-methyl-8-azabicyclo[3.2.1]oct-3-yl)-2-{(4-pentyn-2-yl)oxy}benzamide hydrochloride; (+)-endo-4-amino-5-chloro-N-(8-methyl-8-azabicyclo[3.2.1]oct-3-yl)-2-{(4-pentyn-2-yl)oxy}benzamide; (−)-endo-4-amino-5-chloro-N-(8-methyl-8-azabicyclo[3.2.1]oct-3-yl)-2-{(4-pentyn-2-yl)oxy}benzamide; (+)-endo-4-amino-5-chloro-N-(8-methyl-8-azabicyclo[3.2.1]oct-3-yl)-2-{(4-pentyn-2-yl)oxy}benzamide hydrochloride; (−)-endo-4-amino-5-chloro-N-(8-methyl-8-azabicyclo[3.2.1]oct-3-yl)-2-{(4-pentyn-2-yl)oxy}benzamide hydrochloride; endo-4-acetamido-2-(3-butynyloxy)-5-chloro-N-(8-methyl-8-azabicyclo[3.2.1]oct-3-yl)benzamide; endo-4-amino-2-(3-butynyloxy)-5-chloro-N-(8-methyl-8-azabicyclo[3.2.1]oct-3-yl)benzamide; endo-4-amino-2-(3-butynyloxy)-5-chloro-N-(8-methyl-8-azabicyclo[3.2.1]oct-3-yl)benzamide hydrochloride; (S)-endo-4-amino-5-chloro-N-(8,8-dimethyl-8-azoniabicyclo[3.2.1]oct-3-yl)-2-{(3-pentyn-2-yl)oxy}-benzamide iodide; (S)-endo-4-amino-5-chloro-N-(8-methyl-8-azabicyclo[3.2.1]oct-3-yl)-2-{(3-pentyn-2-yl)oxy}benzamide 8-oxide; (S)-exo-4-amino-5-chloro-N-(8-methyl-8-azabicyclo[3.2.1]oct-3-yl)-2-{(3-pentyn-2-yl)oxy}benzamide; (S)-exo-4-amino-5-chloro-N-(8-methyl-8-azabicyclo[3.2.1]oct-3-yl)-2-{(3-pentyn-2-yl)oxy}benzamide hydrochloride; pharmaceutically acceptable salts thereof and/or stereoisomers thereof.

The pharmaceutically acceptable salt according to the present invention includes inorganic acid salts such as hydrochloride, hydrobromide, sulfate and phosphate; organic acid salts such as acetate, maleate, tartrate, methanesulfonate, benzenesulfonate and toluenesulfonate; and amino acid salts such as argininate, aspartate and glutamate. Further, the aminobenzoic acid compound of the present invention may form a metal salt such as sodium, potassium, calcium or magnesium salt or may be present as a hydrate. The pharmaceutically acceptable salt of the present invention include these metal salts and hydrates. Although the compound of the present invention may be present as geometrical isomers or optical isomers, the invention includes all of the isomers.

The aminobenzoic acid compounds described above can be prepared by processes described in U.S. Pat. No. 5,389,643, the disclosure of which is incorporated by reference herein in its entirety.

In other embodiments, the aminobenzoic acid compounds of the invention are compounds of formula (VIII): 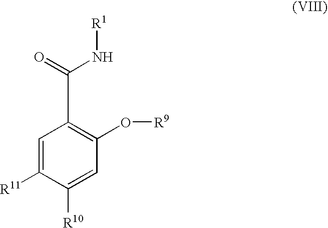
wherein R¹ is 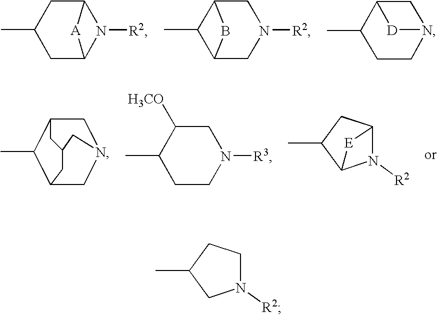
A and B are each independently are —CH₂—X—CH₂— or —CH(R⁸)—CH₂—; X is O, >N—R⁶ or >CHR⁷; R⁶ is a lower alkyl group; R⁷ is hydrogen or a lower alkoxy group; R⁸ is hydrogen, hydroxyl or lower alkoxy; D and E are each independently —(CH₂)₃— or —O—(CH₂)₂—; R² is hydrogen, lower alkyl or arylalkyl, provided that when R⁸ is hydrogen then R² is not methyl; R³ is hydrogen, lower alkyl, —CO₂(CH₂)ₙH or 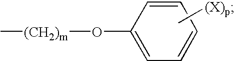
n and m are each independently an integer of 1 to 4; X is halogen, lower alkyl or lower alkoxy; p is 0 or an integer of 1 to 5; R⁹ is alkynyl; R¹⁰ is amino, acylamino or alkylamino; and
R¹¹ is halogen. In other embodiments, the compound of formula (VIII) is in the form of a pharmaceutically acceptable salt; in the form of a stereoisomer(s); or in the form of a pharmaceutically acceptable salt of a stereoisomer(s).

In other embodiments, the aminobenzoic acid compounds of the invention are compounds of Formula (IX): 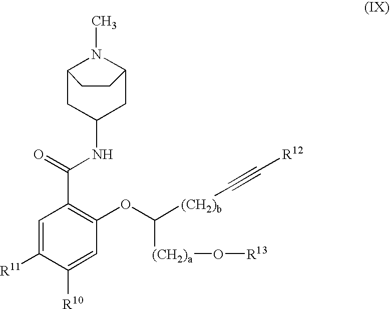
wherein R¹⁰ is amino, acylamino or alkylamino; R¹¹ is halogen; R¹² and R¹³ are each independently a lower alkyl; a is an integer of 1 to 5 (preferably 2); and b is an integer of 0 to 5 (preferably 0). In other embodiments, the compound of formula (IX) is in the form of a pharmaceutically acceptable salt; in the form of a stereoisomer(s); or in the form of a pharmaceutically acceptable salt of a stereoisomer(s).

In Formulae (VIII) and (IX), the lower alkyl defined with respect to R², R³, R⁶, R¹² and R¹³ is a linear or branched one having 1 to 3 carbon atoms, such as methyl, ethyl, propyl or isopropyl. Further, the lower alkoxy defined with respect to R⁷ and R⁸ is methoxy, ethoxy, n-propoxy or isopropoxy. Furthermore, the arylalkyl defined with respect to R² is benzyl, phenylethyl, phenylpropyl or the like. The alkynyl defined with respect to R⁹ is preferably —CH(CH₃)—C≡C—CH₃. The acyl constituting the acylamino defined with respect to R¹⁰ may be one derived from an aliphatic saturated carboxylic acid, an aliphatic unsaturated carboxylic acid, a saturated or unsaturated carbocyclic carboxylic acid, a heterocyclic carboxylic acid, a hydroxycarboxylic acid or any other carboxylic acid. Examples of the acyl include formyl, acetyl, propionyl, benzoyl, toluoyl, naphthoyl, furoyl and nicotinoyl. Further, the alkyl constituting the alkylamino defined with respect to R¹⁰ is as defined above with respect to the lower alkyl. The halogen defined with respect to R¹¹ is chlorine, fluorine, bromine or the like.

Other embodiments of aminobenzoic acid compounds of the invention include the compounds of formula (X), (XI) and (XII):

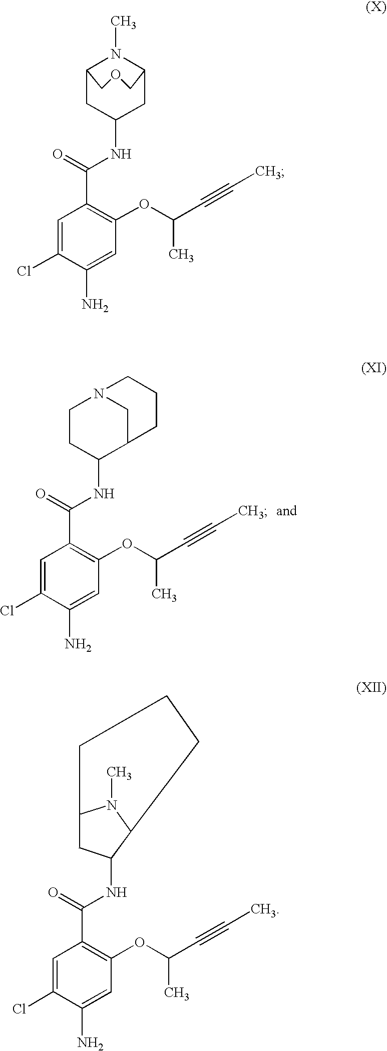

In other embodiments, the compounds of formula (X), (XI) and (XII) are in the form of a pharmaceutically acceptable salt; in the form of a stereoisomer(s); or in the form of a pharmaceutically acceptable salt of a stereoisomer(s).

In other embodiments, the aminobenzoic acid compounds of the invention can be (S)—N-(9-methyl-3-oxa-9-azabicyclo[3.3.1]non-7α-yl)-4-amino-5-chloro-2-(1-methyl-2-butynyl)oxybenzamide; (S)—N-(9-methyl-3-oxa-9-azabicyclo[3.3.1]non-7α-yl)-4-amino-5-chloro-2-(1-methyl-2-butynyl)oxybenzamide hydrochloride; (S)—N-(9-methyl-9-azabicyclo[3.3.1]non-3α-yl)-4-amino-5-chloro-2-(1-methyl-2-butynyl)oxybenzamide; (S)—N-(9-methyl-9-azabicyclo[3.3.1]-non-3α-yl)-4-amino-5-chloro-2-(1-methyl-2-butynyl)oxybenzamide hydrochloride; N-(1-azabicyclo[3.3.1]-non-4α-yl)-4-amino-5-chloro-2-((S)-1-methyl-2-butynyl)oxybenzamide; N-(1-azabicyclo[3.3.1]-non-4α-yl)-4-amino-5-chloro-2-((S)-1-methyl-2-butynyl)oxybenzamide hydrochloride; cis-N-(1-ethoxycarbonyl-3-methoxy-4-piperidinyl)-4-amino-5-chloro-2-((S)-1-methyl-2-butynyl)oxybenzamide; cis-N-(3-methoxy-4-piperidinyl)-4-amino-5-chloro-2-((S)-1-methyl-2-butynyl)oxybenzamide; cis-N-(3-methoxy-4-piperidinyl)-4-amino-5-chloro-2-((S)-1-methyl-2-butynyl)oxybenzamide hydrochloride; cis-N-[1-[3-(4-fluorophenoxy)propyl]-3-methoxy-4-piperidinyl]-4-amino-5-chloro-2-((S)-1-methyl-2-butynyl)oxybenzamide; cis-N-[1-[3-14-fluorophenoxy)propyl]-3-methoxy-4-piperidinyl]-4-amino-5-chloro-2-((S)-1-methyl-2-butynyl)oxybenzamide; cis-N-(1-methyl-3-methoxy-4-piperidinyl)-4-amino-5-chloro-2-((S)-1-methyl-2-butynyl)oxybenzamide; cis-N-(1-methyl-3-methoxy-4-piperidinyl)-4-amino-5-chloro-2-((S)-1-methyl-2-butynyl)oxybenzamide hydrochloride; N-(4-oxa-1-azabicyclo[3.3.1]non-6α-yl)-4-amino-5-chloro-2-((S)-1-methyl-2-butynyl)oxybenzamide; N-(4-oxa-1-azabicyclo[3.3.1]non-6α-yl)-4-amino-5-chloro-2-((S)-1-methyl-2-butynyl)oxybenzamide hydrochloride; N-((6S)-6β-hydroxy-8-methyl-8-azabicyclo[3.2.1]oct-3α-yl)-4-amino-5-chloro-2-((S)-1-methyl-2-butynyl)oxybenzamide; N-((6R)-6β-hydroxy-8-methyl-8-azabicyclo[3.2.1]oct-3α-yl)-4-amino-5-chloro-2-((S)-1-methyl-2-butynyl)oxybenzamide; N-(DL-6α-hydroxy-8-methyl-8-azabicyclo[3.2.1]oct-3α-yl)-4-amino-5-chloro-2-((S)-1-methyl-2-butynyl)oxybenzamide; (S)—N-(3,9-dimethyl-3,9-diazabicyclo[3.3.1]non-7α-yl)-4-amino-5-chloro-2-(1-methyl-2-butynyl)oxybenzamide; (S)—N-(3,9-dimethyl-3,9-diazabicyclo[3.3.1]non-7α-yl)-4-amino-5-chloro-2-(1-methyl-2-butynyl)oxybenzamide dihydrochloride; N-(5S)-4-oxa-1-azabicyclo[3.3.1]non-6α-yl)-4-amino-5-chloro-2-((S)-1-methyl-2-butynyl)oxybenzamide hydrochloride; N-(5R)-4-oxa-1-azabicyclo[3.3.1]non-6α-yl)-4-amino-5-chloro-2-((S)-1-methyl-2-butynyl)oxybenzamide hydrochloride; N-(4-oxa-1-azabicyclo[3.3.1]non-6β-yl)-4-amino-5-chloro-2-((S)-1-methy 1-2-butynyl)oxybenzamide; N-(4-oxa-1-azabicyclo[3.3.1]non-6β-yl)-4-amino-5-chloro-2-((S)-1-methyl-2-butynyl)oxybenzamide hydrochloride; N-((6R)-6β-methoxy-8-methyl-8-azabicyclo[3.2.1]oct-3β-yl)-4-amino-5-chloro-2-((S)-1-methyl-2-butynyl)oxybenzamide; N-((6S)-6β-methoxy-8-methyl-8-azabicyclo[3.2.1]oct-3β-yl)-4-amino-5-chloro-2-((S)-1-methyl-2-butynyl)oxybenzamide; N-((6S)-6β-methoxy-8-methyl-8-azabicyclo[3.2.1]oct-3β-yl)-4-amino-5-chloro-2-((S)-1-methyl-2-butynyl)oxybenzamide hydrochloride; N-((6R)-6β-methoxy-8-methyl-8-azabicyclo[3.2.1]oct-3β-yl)-4-amino-5-chloro-2-((S)-1-methyl-2-butynyl)oxybenzamide hydrochloride; N-(6α-methoxy-8-methyl-8-azabicyclo[3.2.1]oct-3α-yl)-4-amino-5-chloro-2-((S)-1-methyl-2-butynyl)oxybenzamide; N-(6α-methoxy-8-methyl-8-azabicyclo[3.2.1]oct-3α-yl)-4-amino-5-chloro-2-((S)-1-methyl-2-butynyl)oxybenzamide; N-((6S)-6β-methoxy-8-methyl-8-azabicyclo[3.2.1]oct-3α-yl)-4-amino-5-chloro-2-((S)-1-methyl-2-butynyl)oxybenzamide; N-((6R)-6β-methoxy-8-methyl-8-azabicyclo[3.2.1]oct-3α-yl)-4-amino-5-chloro-2-((S)-1-methyl-2-butynyl)oxybenzamide; N-((6S)-6β-methoxy-8-methyl-8-azabicyclo[3.2.1]oct-3α-yl)-4-amino-5-chloro-2-((S)-1-methyl-2-butynyl)oxybenzamide hydrochloride; N-((6R)-6β-methoxy-8-methyl-8-azabicyclo[3.2.1]oct-3α-yl)-4-amino-5-chloro-2-((S)-1-methyl-2-butynyl)oxybenzamide hydrochloride; N-(8-azabicyclo[3.2.1]oct-3α-yl)-4-amino-5-chloro-2-((S)-1-methyl-2-butynyl)oxybenzamide; N-((5R)-1-azabicyclo[3.3.1]non-4α-yl)-4-amino-5-chloro-2-((S)-1-methyl-2-butynyl)oxybenzamide; N-((5S)-1-azabicyclo[3.3.1]non-4α-yl)-4-amino-5-chloro-2-((S)-1-methyl-2-butynyl)oxybenzamide; N-((5R)-1-azabicyclo[3.3.1]non-4α-yl)-4-amino-5-chloro-2-((S)-1-methyl-2-butynyl)oxybenzamide hydrochloride; N-((5S)-1-azabicyclo[3.3.1]non-4α-yl)-4-amino-5-chloro-2-((S)-1-methyl-2-butynyl)oxybenzamide hydrochloride; N-(1-azabicyclo[3.3.1]non-4β-yl)-4-amino-5-chloro-2-((S)-1-methyl-2-butynyl)oxybenzamide; N-(1-azabicyclo[3.3.1]non-4β-yl)-4-amino-5-chloro-2-((S)-1-methyl-2-butynyl)oxybenzamide hydrochloride; (S)—N-(3-methyl-3-azabicyclo[3.2.1]oct-8α-yl)-4-amino-5-chloro-2-(1-methyl-2-butynyl)oxybenzamide; (S)—N-(3-methyl-3-azabicyclo[3.2.1]oct-8β-yl)-4-amino-5-chloro-2-(1-methyl-2-butynyl)oxybenzamide; (S)—N-(3-methyl-3-azabicyclo[3.2.1]oct-8α-yl)-4-amino-5-chloro-2-(1-methyl-2-butynyl)oxybenzamide hydrochloride; (S)—N-(3-methyl-3-azabicyclo[3.2.1]oct-8β-yl)-4-amino-5-chloro-2-(1-methyl-2-butynyl)oxybenzamide hydrochloride; (S)—N-(3-isopropyl-3-azabicyclo[3.2.1]oct-8-yl)-4-amino-5-chloro-2-(1-methy 1-2-butynyl)oxybenzamide; (S)—N-(3-isopropyl-3-azabicyclo[3.2.1]oct-8-yl)-4-amino-5-chloro-2-(1-methy 1-2-butynyl)oxybenzamide hydrochloride; (S)—N-(3-benzyl-3-azabicyclo[3.2.1]oct-8-yl)-4-amino-5-chloro-2-(1-methyl-2-butynyl)oxybenzamide; (S)—N-(3-benzyl-3-azabicyclo[3.2.1]oct-8-yl)-4-amino-5-chloro-2-(1-methyl-2-butynyl)oxybenzamide hydrochloride; N-(8-methyl-8-azabicyclo[3.2.1]oct-3α-yl)-4-amino-5-chloro-2-(1-methyl-2-butynyl)oxybenzamide; N-(8-methyl-8-azabicyclo[3.2.1]oct-3α-yl)-4-amino-5-chloro-2-(1-methyl-2-butynyl)oxybenzamide hydrochloride; (S)—N-(3,7-dimethyl-3,7-diazabicyclo[3.3.1]non-9-yl)-4-amino-5-chloro-2-(1-methyl-2-butynyl)oxybenzamide; (S)—N-(3,7-dimethyl-3,7-diazabicyclo[3.3.1]non-9-yl)-4-amino-5-chloro-2-(1-methyl-2-butynyl)oxybenzamide hydrochloride; (S)—N-(1-benzyl-3-pyrrolidyl)-4-amino-5-chloro-2-(1-methyl-2-butynyl)oxybenzamide; (S)—N-(1-benzyl-3-pyrrolidyl)-4-amino-5-chloro-2-(1-methyl-2-butynyl)oxybenzamide hydrochloride; (S)—N-(3-oxa-7-methyl-7-azabicyclo[3.3.1]non-9α-yl)-4-amino-5-chloro-2-(1-methyl-2-butynyl)oxybenzamide; (S)—N-(3-oxa-7-methyl-7-azabicyclo[3.3.1]non-9β-yl)-4-amino-5-chloro-2-(1-methyl-2-butynyl)oxybenzamide; (S)—N-(3-oxa-7-methyl-7-azabicyclo[3.3.1]non-9α-yl)-4-amino-5-chloro-2-(1-methyl-2-butynyl)oxybenzamide hydrochloride; (S)—N-(3-oxa-7-methyl-7-azabicyclo[3.3.1]non-9β-yl)-4-amino-5-chloro-2-(1-methyl-2-butynyl)oxybenzamide hydrochloride; (S)—N-(3-ethoxy-9-methyl-9-azabicyclo[3.3.1]non-9α-yl)-4-amino-5-chloro-2-(1-methyl-2-butynyl)oxybenzamide; (S)—N-(3-ethoxy-9-methyl-9-azabicyclo[3.3.1]non-9β-yl)-4-amino-5-chloro-2-(1-methyl-2-butynyl)oxybenzamide; (S)—N-(3-ethoxy-9-methyl-9-azabicyclo[3.3.1]non-9α-yl)-4-amino-5-chloro-2-(1-methyl-2-butynyl)oxybenzamide hydrochloride; (S)—N-(3-ethoxy-9-methyl-9-azabicyclo[3.3.1]non-9β-yl)-4-amino-5-chloro-2-(1-methyl-2-butynyl)oxybenzamide hydrochloride; (S)—N-(8-methyl-8-azabicyclo[3.2.1]oct-2α-yl)-4-amino-5-chloro-2-(1-methyl-2-butynyl)oxybenzamide; (S)—N-(8-methyl-8-azabicyclo[3.2.1]oct-2β-yl)-4-amino-5-chloro-2-(1-methyl-2-butynyl)oxybenzamide; (S)—N-(8-methyl-8-azabicyclo[3.2.1]oct-2α-yl)-4-amino-5-chloro-2-(1-methyl-2-butynyl)oxybenzamide hydrochloride; (S)—N-(8-methyl-8-azabicyclo[3.2.1]oct-2β-yl)-4-amino-5-chloro-2-(1-methyl-2-butynyl)oxybenzamide hydrochloride; (S)—N-(1-azatricyclo[3.3.1.1^{3,7}]dec-10-yl)-4-amino-5-chloro-(1-methyl-2-butynyl)oxybenzamide; and (S)—N-(1-azatricyclo[3.3.1.1^{3,7}]dec-10-yl)-4-amino-5-chloro-(1-methyl-2-butynyl)oxybenzamide hydrochloride; pharmaceutically acceptable salts thereof and/or stereoisomers thereof.

The pharmaceutically acceptable salt according to the present invention includes inorganic acid salts such as hydrochloride, hydrobromide, sulfate and phosphate; organic acid salts such as acetate, maleate, tartrate, methanesulfonate, benzenesulfonate and toluenesulfonate; and amino acid salts such as argininate, aspartate and glutamate. Further, the aminobenzoic acid compound of the present invention may form a metal salt such as sodium, potassium, calcium or magnesium salt or may be present as a hydrate. The pharmaceutically acceptable salt of the present invention include these metal salts and hydrates. Although the compound of the present invention may be present as geometrical isomers or optical isomers, the invention includes all of the isomers.

The aminobenzoic acid compounds described above can be prepared by processes described in U.S. Pat. Nos. 5,512,579, 5,658,925, 5,677,311, 5,723,472 and 5,861,418, the disclosures of which are incorporated by reference herein in their entirety.

The compounds of the invention can be administered as any pharmaceutically acceptable salt known in the art. Pharmaceutically acceptable salts are known in the art and include those of inorganic acids, such as hydrochloride, sulfate, hydrobromide, sulfate, and phosphate; those of organic acids, such as formate, acetate, maleate, tartrate, trifluoroacetate, methanesulfonate, benzenesulfonate and toluenesulfonate, and those of amino acids such as arginine, aspartic acid and glutamic acid. When certain substituents are selected, the compounds of the invention can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the compounds of the invention can be made in the form of any of these or of any other pharmaceutically acceptable salt.

A therapeutically effective dosage regimen for treating the diseases described herein with the aminobenzoic acid compounds and optionally, proton pump inhibitors, cholinesterase inhibitors, histamine agonists, antacids, bismuth compounds, sucralfate, cisapride, misoprostol, NSAIDs, steroids, VIP, secretin, vitamins, cholinergic compounds and/or migraine drugs is selected in accordance with a variety of factors, including the age, weight, sex, and medical condition of the patient, the severity of the disease, the route of administration, pharmacological considerations such as the activity, efficacy, pharmacokinetic and toxicology profiles of the particular drugs, whether a drug delivery system is used and whether the drugs are administered as part of a drug combination.

When administered separately, the aminobenzoic acid compounds and, optionally, proton pump inhibitors, cholinesterase inhibitors, histamine agonists, antacids, bismuth compounds, sucralfate, cisapride, misoprostol, NSAIDs, VIP, secretin, vitamins, steroids, cholinergic compounds, and/or migraine drugs, can be administered about the same time as part of an overall treatment regimen, i.e., as a therapeutic cocktail or a combination therapy. “About the same time” includes administering the aminobenzoic acid compound and/or histamine agonists, proton pump inhibitors, cholinesterase inhibitors, antacids, bismuth compounds, sucralfate, cisapride, misoprostol, NSAIDs, steroids, cholinergic compounds, VIP, secretin, vitamins, and/or migraine drugs at the same time, at different times on the same day, or on different days, as long as they are administered as part of an overall treatment regimen.

The aminobenzoic acid compounds can be administered in amounts of about 0.01 to about 1000 mg per day, preferably about 0.1 to about 500 mg per day, more preferably about 0.1 to about 100 mg per day. When the compound is administered as an injection, the dose is generally 1 to 3000 μg/kg, preferably about 3 to 1000 μg/kg. The compounds and/or compositions can be administered once a day or in divided doses, for example from 2 to 4 times a day, preferably once per day. One skilled in the art will recognize that when the compounds and/or compositions of the invention are administered to infants or children, the dose can be smaller than the dose administered to adults, and that the dose can be dependent upon the size and weight of the patient.

The proton pump inhibitors, cholinesterase inhibitors, histamine antagonists, antacids, bismuth compounds, sucralfate, cisapride, misoprostol, NSAIDs, steroids, VIP, secretin, vitamins, cholinergic compounds, and migraine drugs can be prepared by processes known in the art or can be obtained from commercial sources, and can be administered in therapeutically effective doses that are known in the art, such as those described in the Physician's Desk Reference.

The aminobenzoic acid compounds and/or proton pump inhibitors, cholinesterase inhibitors histamine agonists, antacids, bismuth compounds, sucralfate, cisapride, misoprostol, NSAIDs, steroids, VIP, secretin, vitamins, cholinergic compounds, and/or migraine drugs can be administered orally, topically, parenterally, by inhalation (nasal, oral, aural), or rectally in dosage unit formulations containing conventional nontoxic pharmaceutically acceptable carriers, adjuvants, and vehicles as desired. The term parenteral as used herein includes subcutaneous, intravenous, intramuscular, intrathecal, intrasternal injection, or infusion techniques. Preferably, the aminobenzoic acid compounds are orally administered as tablets.

Injectable preparations, for example, sterile injectable aqueous or oleaginous suspensions can be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), preservatives and/or stabilizers. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids such as oleic acid find use in the preparation of injectables. The preparations can be lyophilized by methods known in the art.

Solid dosage forms for oral administration can include capsules, tablets, sublingual tablets, powders, granules, gels, effervescent tablets, effervescent wafers, effervescent capsules, and effervescent powders; most preferably tablets. The solid dosage form can be a solid microencapsulated dosage, such as a microencapsulated powder, microencapsulated granules or a microencapsulated gel. A solid dosage form for oral administration can be prepared by mixing an active principle with filler and, if necessary, binder, disintegrating agent, lubricant, coloring agent, corrigent or the like and converting the obtained mixture into a tablet, coated tablet, granule, powder or capsule. Examples of the filler include lactose, corn starch, sucrose, glucose, sorbitol, crystalline cellulose and silicon dioxide, while those of the binder include polyvinyl alcohol, polyvinyl ether, ethylcellulose, methylcellulose, acacia, tragacanth, gelatin, shellac, hydroxypropylcellulose, hydroxypropylstarch and polyvinylpyrrolidone. Examples of the disintegrating agent include starch, agar, gelatin powder, crystalline cellulose, calcium carbonate, sodium hydrogencarbonate, calcium citrate, dextrin and pectin, while those of the lubricant include magnesium stearate, talc, polyethylene glycol, silica and hardened vegetable oils. The coloring agent can be any one which is permitted to be added to drugs. Examples of the corrigent include cacao powder, mentha herb, aromatic powder, mentha oil, borneol and powdered cinnamon bark. The tablets and granules can be, if necessary, coated with sugar, gelatin or the like. Preferably, the tablets have an enteric coating.

The aminobenzoic acid compound can be formulated or admixed with, for example, an acid (e.g., citric acid) and a bicarbonate (e.g., sodium bicarbonate) to form an effervescent tablet, capsule, wafer or powder. After addition of the effervescent tablet, capsule, wafer or powder to a liquid (e.g., water, juice) a bicarbonate solution of the aminobenzoic acid compound is formed.

In other embodiments, the solid dosage form can be packaged as granules or a powder in a pharmaceutically acceptable carrier, where the granules or powder are removed from the packaging and sprinkled on food or mixed with a liquid, such as water or juice. In this embodiment, the active compound can be mixed with flavoring or sweetening agents. The packaging material can be plastic, polyester films, nylon films, polyolefin films, shrink packing films, coated paper, or any material that prevents water or moisture from reaching the granules and/or powder.

Liquid dosage forms for oral administration can include pharmaceutically acceptable emulsions, solutions, suspensions, and syrups containing inert diluents commonly used in the art, such as water. The liquid dosage form can be a microencapsulated liquid, including microencapsulated emulsions, microencapsulated solutions, microencapsulated suspensions and microencapsulated syrups. Such compositions can also comprise adjuvants, such as wetting agents, emulsifying and suspending agents, and sweetening, flavoring, and perfuming agents.

In another embodiment, the invention provides compositions comprising at least one aminobenzoic acid compound and at least one cyclodextrin and/or cyclodextrin compound. The compositions can be in the form of a sachet, granules, micro-pellets, or beads. Cyclodextrin compounds are described, for example, in U.S. Pat. No. 3,459,731, EP-A-149,197, EP-A-197,571, U.S. Pat. No. 4,535,152 or WO 90/12035. Cyclodextrin compounds include alpha-cyclodextrins, beta-cyclodextrins, and gamma-cyclodextrins. The cyclodextrins can be ethers and/or mixed ethers thereof wherein one or more of the hydroxy groups of the anhydroglucose units of the cyclodextrin are substituted with C₁₋₆ alkyl (e.g., methyl, ethyl or isopropyl); hydroxy C₁₋₆ alkyl (e.g., hydroxyethyl, hydroxypropyl or hydroxybutyl); carboxy C₁₋₆ alkyl (e.g., carboxymethyl or carboxyethyl); C₁₋₆ alkyl-carbonyl (e.g., acetyl); C₁₋₆ alkyloxycarbonyl C₁₋₆ alkyl or carboxy C₁₋₆ alkyl-oxy C₁₋₆ alkyl (e.g., carboxymethoxypropyl or carboxyethoxypropyl); C₁₋₆ alkylcarbonyloxy C₁₋₆ alkyl (e.g., 2-acetyloxypropyl). In one embodiment, complexants and/or solubilizers for the aminobenzoic acid compounds are beta-cyclodextrin; 2,6-dimethyl-beta-cyclodextrin, 2-hydroxyethyl-beta-cyclodextrin, 2-hydroxyethyl-gamma-cyclodextrin, 2-hydroxypropyl-gamma-cyclodextrin and (2-carboxy-methoxy)propyl-beta-cyclodextrin. and in particular 2-hydroxypropyl-beta-cyclodextrin. In another embodiment, the cyclodextrin is beta-cyclodextrin.

For administration by aural, oral or nasal inhalation, the compounds and compositions can be delivered from an insufflator, a nebulizer or a pressured pack or other convenient mode of delivering an aerosol spray. Pressurized packs can include a suitable propellant. Alternatively, for administration by aural, oral or nasal inhalation, the compounds and compositions can be administered in the form of a dry powder composition or in the form of a liquid spray or drop.

Suppositories for rectal administration can be prepared by mixing one or more compounds or compositions with suitable nonirritating excipients, such as cocoa butter and/or polyethylene glycols, that are solid at room temperature and that melt at body temperature. Alternatively, enemas can be used to for rectal administration of the active compounds.

For topical administration to the epidermis, the aminobenzoic acid compounds can be formulated as ointments, creams or lotions, or as the active ingredient of a transdermal patch. The compounds and compositions can also be administered via iontophoresis or osmotic pump. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the aminobenzoic acid compounds can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrable compositions can contain polyethylene glycol 400. To form ointments, the aminobenzoic acid compounds can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The aminobenzoic acid compounds can also be topically applied using a transdermal system, such as one of an acrylic-based polymer adhesive with a resinous crosslinking agent impregnated with the aminobenzoic acid compounds and laminated to an impermeable backing. For example, the aminobenzoic acid compounds can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

The invention provides for the aminobenzoic acid compounds and, optionally, other active ingredients, to be administered nasally to a patient to treat the diseases and disorders described herein and those described, for example, in PCT Application No. PCT/US02/36857, the disclosure of which is incorporated by reference herein in its entirety. “Administered nasally” or “nasal administration” is intended to mean that at least one aminobenzoic acid compound is combined with a suitable delivery system for absorption across the nasal mucosa of a patient, preferably a human.

The aminobenzoic acid compounds of the invention can be administered, for example, as nasal sprays, nasal drops, nasal suspensions, nasal gels, nasal ointments, nasal creams or nasal powders. The aminobenzoic acid compounds can also be administered using nasal tampons or nasal sponges. The aminobenzoic acid compounds of the invention can be brought into a viscous basis via systems conventionally used, for example, natural gums, methylcellulose and compounds, acrylic polymers (carbopol) and vinyl polymers (polyvinylpyrrolidone). In the compositions, many other excipients known in the art can be added such as water, preservatives, surfactants, solvents, adhesives, antioxidants, buffers, bio-adhesives, viscosity enhancing agents and agents to adjust the pH and the osmolarity.

The nasal delivery systems can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In other embodiments, the nasal delivery system can be a powder formulation. Powder formulations include, for example, powder mixtures, powder microspheres, coated powder microspheres, liposomal dispersions and combinations thereof. Preferably, the powder formulation is powder microspheres. The powder microspheres are preferably formed from various polysaccharides and celluloses selected from starch, methylcellulose, xanthan gum, carboxymethylcellulose, hydroxypropyl cellulose, carbomer, alginate polyvinyl alcohol, acacia, chitosans, and mixtures of two or more thereof.

In certain embodiments, the particle size of the droplets of the aqueous and/or non-aqueous solution or of the powders delivered to the nasal mucosa can be, for example, about 0.1 micron to about 100 microns; from about 1 micron to about 70 microns; from about 5 microns to about 50 microns; or from about 10 microns to about 20 microns. The particle sizes can be obtained using suitable containers or metering devices known in the art. Exemplary devices include mechanical pumps in which delivery is made by movement of a piston; compressed air mechanisms in which delivery is made by hand pumping air into the container; compressed gas (e.g., nitrogen) techniques in which delivery is made by the controlled release of a compressed gas in the sealed container; liquefied propellant techniques in which a low boiling liquid hydrocarbon (e.g., butane) is vaporized to exert a pressure and force the composition through the metered valve; and the like. Powders may be administered, for example, in such a manner that they are placed in a capsule that is then set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In one embodiment, the invention provides a nasally administrable pharmaceutical composition comprising at least one aminobenzoic acid compound dispersed in a nasal delivery system that improves the solubility of the aminobenzoic acid compound. The nasal delivery system that improves solubility can include one of the following or combinations thereof: (i) a glycol compound (e.g., propylene glycol, polyethylene glycol, mixtures thereof); (ii) a sugar alcohol (e.g., mannitol, xylitol, mixtures thereof); (iii) glycerin; (iv) a glycol compound (e.g., propylene glycol, polyethylene glycol or mixtures thereof) and glycerin; (v) ascorbic acid and water; (vi) sodium ascorbate and water; or (vii) sodium metabisulfite and water.

In another embodiment, the invention provides a nasally administrable pharmaceutical composition comprising at least one aminobenzoic acid compound and a nasal delivery system, where the nasal delivery system comprises at least one buffer to maintain the pH of the aminobenzoic acid compound, at least one pharmaceutically acceptable thickening agent and at least one humectant. The nasal delivery system can optionally further comprise surfactants, preservatives, antioxidants, bio-adhesives, pH adjusting agents, isotonicity agents, solubilizing agents, and/or other pharmaceutically acceptable excipients. The aminobenzoic acid compound can optionally be dispersed in a nasal delivery system that improves its solubility.

In another embodiment, the invention provides a nasally administrable pharmaceutical composition comprising at least one aminobenzoic acid compound and a nasal delivery system, where the nasal delivery system comprises at least one solubilizing agent, at least one pharmaceutically acceptable thickening agent and at least one humectant. The nasal delivery system can optionally further comprise buffers, pH adjusting agents, isotonicity agents, surfactants, preservatives, antioxidants, bio-adhesives, and/or other pharmaceutically acceptable excipients. The aminobenzoic acid compound can optionally be dispersed in a nasal delivery system that improves its solubility.

In another embodiment, the invention provides a nasally administrable pharmaceutical composition comprising at least one aminobenzoic acid compound and a nasal delivery system, where the nasal delivery system comprises at least one buffer to maintain the pH of the aminobenzoic acid compound, at least one pharmaceutically acceptable thickening agent, at least one humectant, and at least one surfactant. The nasal delivery system can optionally further comprise pH adjusting agents, isotonicity agents, solubilizing agents, preservatives, antioxidants, bio-adhesives, and/or other pharmaceutically acceptable excipients. The aminobenzoic acid compound can optionally be dispersed in a nasal delivery system that improves its solubility.

In yet another embodiment, the invention provides a nasally administrable pharmaceutical composition comprising at least one aminobenzoic acid compound and a nasal delivery system, where the nasal delivery system comprises at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The aminobenzoic acid compound can optionally be dispersed in a nasal delivery system that improves its solubility.

In yet another embodiment, the invention provides a nasally administrable pharmaceutical composition comprising at least one aminobenzoic acid compound and a nasal delivery system, where the nasal delivery system comprises at least one buffer to maintain the pH of the aminobenzoic acid compound, at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The aminobenzoic acid compound can optionally be dispersed in a nasal delivery system that improves its solubility.

The nasally administrable pharmaceutical compositions of the invention preferably provide a peak plasma concentration of the aminobenzoic acid compound in less than one hour, preferably within about 5 minutes to about 30 minutes, more preferably within about 5 minutes to about 20 minutes, after administration to the patient.

The buffer has a pH that is selected to optimize the absorption of the aminobenzoic acid compound across the nasal mucosa. The particular pH of the buffer can vary depending upon the particular nasal delivery formulation as well as the specific aminobenzoic acid compound selected. Buffers that are suitable for use in the invention include acetate (e.g., sodium acetate), citrate (e.g., sodium citrate dihydrate), phthalate, borate, prolamine, trolamine, carbonate, phosphate (e.g., monopotassium phosphate, disodium phosphate), and mixtures of two or more thereof.

The pH of the compositions should be maintained from about 3.0 to about 10.0. Compositions having a pH of less than about 3.0 or greater than about 10.0 can increase the risk of irritating the nasal mucosa of the patient. Further, it is preferable that the pH of the compositions be maintained from about 3.0 to about 9.0. With respect to the non-aqueous nasal formulations, suitable forms of buffering agents can be selected such that when the formulation is delivered into the nasal cavity of a mammal, selected pH ranges are achieved therein upon contact with, e.g., a nasal mucosa.

The solubilizing agent for use in the compositions of the invention can be any known in the art, such as carboxylic acids and salts thereof. Exemplary carboxylic acid salts include acetate, gluconate, ascorbate, citrate, fumurate, lactate, tartrate, malate, maleate, succinate, or mixtures of two or more thereof.

The viscosity of the compositions of the present invention can be maintained at a desired level using a pharmaceutically acceptable thickening agent. For example, the viscosity may be at least 1000 cps; from about 1000 to about 10,000 cps; from about 2000 cps to about 6500 cps; or from about 2500 cps to about 5000 cps. Thickening agents that can be used in accordance with the present invention include, for example, methyl cellulose, xanthan gum, carboxymethyl cellulose, hydroxypropyl cellulose, carbomer, polyvinyl alcohol, alginates, acacia, chitosans, and mixtures of two or more thereof. The concentration of the thickening agent will depend upon the agent selected and the viscosity desired. Such agents can also be used in a powder formulation.

The nasally administrable compositions can also include a humectant to reduce or prevent drying of the mucus membrane and to prevent irritation thereof. Suitable humectants that can be used include, for example, sorbitol, mineral oil, vegetable oil and glycerol; soothing agents; membrane conditioners; sweeteners; and mixtures of two or more thereof. The concentration of the humectant will vary depending upon the agent selected. In one embodiment, the humectant can be present in the nasal delivery system in a concentration ranging from about 0.01% to about 20% by weight of the composition.

In other embodiments, the nasal delivery system can further comprise surfactants which enhance the absorption of the aminobenzoic acid compound. Suitable surfactants include non-ionic, anionic and cationic surfactants. Exemplary surfactants include oleic acid, polyoxyethylene compounds of fatty acids, partial esters of sorbitol anhydride, such as for example, Tweens (e.g., Tween 80, Tween 40, Tween 20), Spans (e.g., Span 40, Span 80, Span 20), polyoxyl 40 stearate, polyoxy ethylene 50 stearate, fusieates, bile salts, octoxynol, and mixtures of two or more thereof. Exemplary anionic surfactants include salts of long chain hydrocarbons (e.g., C₆₋₃₀ or C₁₀₋₂₀) having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetradecyl sulfate. One particularly preferred anionic surfactant is sodium lauryl sulfate (i.e., sodium dodecyl sulfate). The surfactants can be present in an amount from about 0.001% to about 50% by weight, or from about 0.001% to about 20% by weight.

The pharmaceutical compositions of the invention may further comprise an isotonicity agent, such as sodium chloride, dextrose, boric acid, sodium tartrate or other inorganic or organic solutes.

The nasal pharmaceutical compositions of the invention can optionally be used in combination with a pH adjusting agent. Exemplary pH adjusting agents include sulfuric acid, sodium hydroxide, hydrochloric acid, and the like.

To extend shelf life, preservatives can be added to the nasally administrable compositions. Suitable preservatives that can be used include benzyl alcohol, parabens, thimerosal, chlorobutanol, benzalkonium chloride, or mixtures of two or more thereof. Preferably benzalkonium chloride is used. Typically, the preservative will be present in a concentration of up to about 2% by weight. The exact concentration of the preservative, however, will vary depending upon the intended use and can be easily ascertained by one skilled in the art.

Other ingredients which extend shelf life can be added such as for example, antioxidants. Some examples of antioxidants include sodium metabisulfite, potassium metabisulfite, ascorbyl palmitate and the like. Typically, the antioxidant will be present in the compositions in a concentration of from about 0.001% up to about 5% by weight of the total composition.

Other optional ingredients can also be incorporated into the nasal delivery system provided that they do not interfere with the action of the aminobenzoic acid compound or significantly decrease the absorption of the aminobenzoic acid compound across the nasal mucosa.

The nasal delivery systems can be made following the processes described in, for example, U.S. Pat. Nos. 6,451,848, 6,436,950, and 5,874,450, and WO 00/00199, the disclosures of which are incorporated by reference herein in their entirety.

The invention provides pharmaceutical kits comprising one or more containers filled with one or more of the ingredients of the pharmaceutical compounds and/or compositions of the invention, including, one or more aminobenzoic acid compounds (e.g., rabeprazole, stereoisomers thereof and/or pharmaceutically acceptable salts thereof) and/or histamine antagonists, antacids, bismuth compounds, sucralfate, cisapride, misoprostol, NSAIDs, migraine drugs, VIP, secretin, and/or vitamins. The aminobenzoic acid compounds and/or histamine antagonists, antacids, bismuth compounds, sucralfate, cisapride, misoprostol, NSAIDs, steroids, cholinergic compounds, VIP, secretin, vitamins, and/or migraine drugs can be separate components in the kit or can be in the form of a composition in the kit. The kits can also include, for example, other compounds and/or compositions, a device(s) for administering the compounds and/or compositions, and written instructions in a form prescribed by a governmental agency regulating the manufacture, use or sale of pharmaceuticals.

While the aminobenzoic acid compounds of the invention can be administered as the sole active pharmaceutical agent in the methods described herein, they can also be used in combination with one or more compounds which are known to be therapeutically effective against the specific disease that one is targeting for treatment.

Each of the patents and publications cited herein are incorporated by reference herein in their entirety. It will be apparent to one skilled in the art that various modifications can be made to the invention without departing from the spirit or scope of the appended claims.

What is claimed is:

1. A method for treating a gastrointestinal disorder, a psychiatric disorder, a learning disability, a sleep disorder, Tourette's syndrome, obesity, epilepsy, post-menopausal syndrome, premenstrual syndrome, asthma, laryngitis, or a migraine in a patient in need thereof comprising administering to the patient a therapeutically effective amount of a compound of Formula (I) or a pharmaceutically acceptable salt thereof, a compound of Formula (II) or a pharmaceutically acceptable salt thereof, a compound of Formula (III) or a pharmaceutically acceptable salt thereof, a compound of Formula (IV) or a pharmaceutically acceptable salt thereof, a compound of Formula (V) or a pharmaceutically acceptable salt thereof, a compound of Formula (VI) or a pharmaceutically acceptable salt thereof, a compound of Formula (VII) or a pharmaceutically acceptable salt thereof, a compound of Formula (VIII) or a pharmaceutically acceptable salt thereof, a compound of Formula (IX) or a pharmaceutically acceptable salt thereof, a compound of Formula (X) or a pharmaceutically acceptable salt thereof, a compound of Formula (XI) or a pharmaceutically acceptable salt thereof, or a compound of Formula (XII) or a pharmaceutically acceptable salt thereof;
wherein the compound of Formula (I) is: 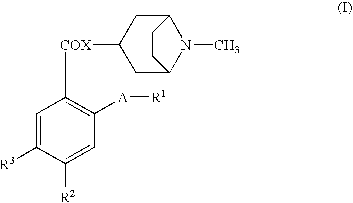
wherein R¹ is a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² is an amino, acylamino, carboxyamino or alkylamino group; R³ is a halogen atom; X is —O— or —NH—; and A is O or S;
wherein the compound of Formula (II) is: 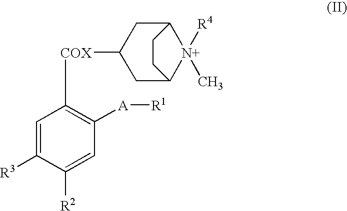
wherein R¹ is a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² is an amino, acylamino, carboxyamino or alkylamino group; R³ is a halogen atom; X is: —O— or —NH—; A is O or S; and R⁴ is a lower alkyl or aralkyl group;
wherein the compound of Formula (III) is: 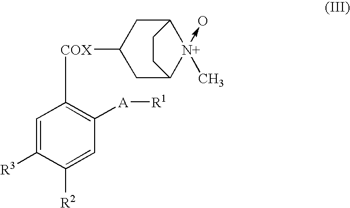
wherein R¹ is a cyanoalkyl, cyanoalkenyl, substituted cycloalkyl, substituted or unsubstituted cycloalkylalkyl, halogen-substituted lower alkyl, alkenyl, alkynyl, saturated or unsaturated heterocyclicalkyl or saturated or unsaturated heterocyclic group; R² is an amino, acylamino, carboxyamino or alkylamino group; R³ is a halogen atom; X is —O— or —NH—; and A is an oxygen atom or a sulfur atom;
wherein the compound of Formula (IV) is: 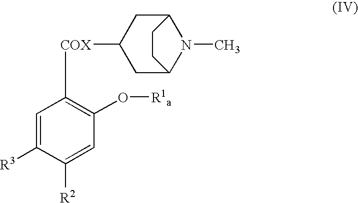
wherein R¹ₐ is an alkynyl group; R² is an amino, acylamino, carboxyamino or alkylamino group; R³ is a halogen atom; and X is —O— or —NH—;
wherein the compound of Formula (V) is: 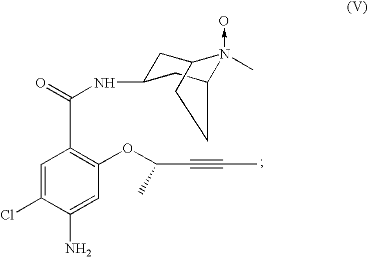
wherein the compound of Formula (VI) is: 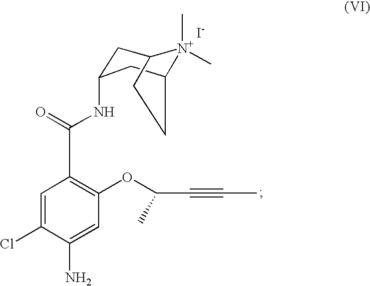
wherein the compound of Formula (VII) is: 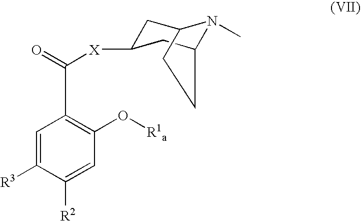
wherein R¹ₐ is an alkynyl group; and X, R² and R³ are each as defined above;
wherein the compound of Formula (VIII) is: 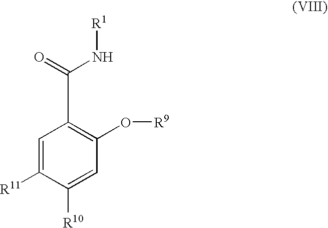
wherein R¹ is 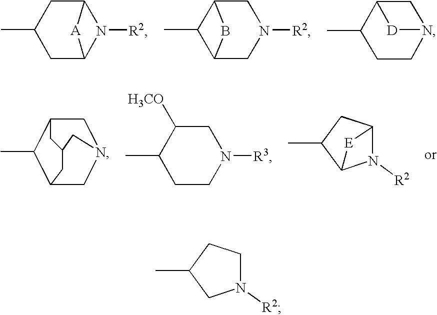
A and B are each independently are —CH₂—X—CH₂— or —CH(R⁸)—CH₂—; X is O, >N—R⁶ or >CHR⁷; R⁶ is a lower alkyl group; R⁷ is hydrogen or a lower alkoxy group; R⁸ is hydrogen, hydroxyl or lower alkoxy; D and E are each independently —(CH₂)₃— or —O—(CH₂)₂—; R² is hydrogen, lower alkyl or arylalkyl, provided that when R⁸ is hydrogen then R² is not methyl; R³ is hydrogen, lower alkyl, —CO₂(CH₂)ₙH or 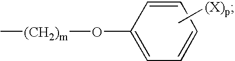
n and m are each independently an integer of 1 to 4; X is halogen, lower alkyl or lower alkoxy; p is 0 or an integer of 1 to 5; R⁹ is alkynyl; R¹⁰ is amino, acylamino or alkylamino; and
R¹¹ is halogen;
wherein the compound of Formula (IX) is: 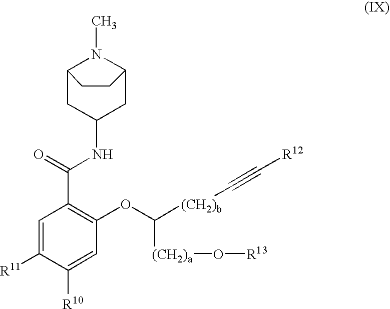
wherein R¹⁰ is amino, acylamino or alkylamino; R¹¹ is halogen; R¹² and R¹³ are each independently a lower alkyl; a is an integer of 1 to 5; and b is an integer of 0 to 5;
wherein the compound of Formula (X) is: 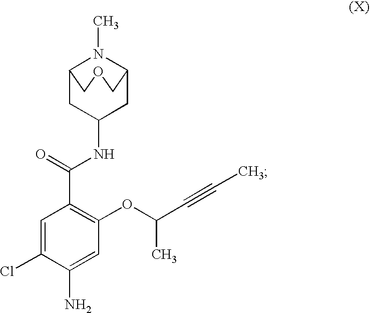
wherein the compound of Formula (XI) is: 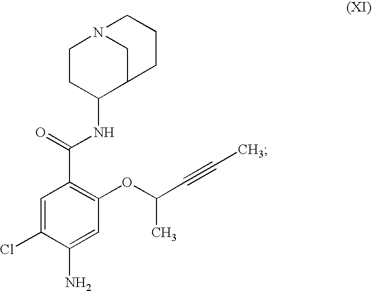
and
wherein the compound of Formula (XII) is:

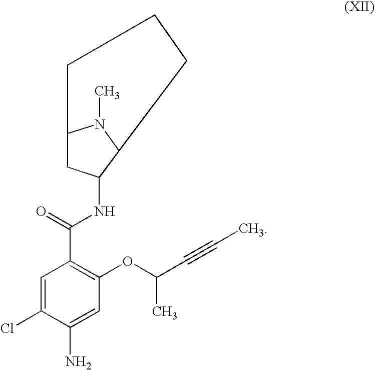

2. The method of claim 1, wherein the gastrointestinal disorder is an ulcer, post-operative aspiration, dyspepsia, acute gastrointestinal bleeding, a lower esophageal mucosal ring, an esophageal stricture, esophageal dismotility, a hiatial hernia, Barrett's esophagus, diverticulosis, diverticulitis, a malabsorption syndrome, gastroesophageal reflux disease, inflammatory bowel disease, infectious enteritis, idiopathic gastric acid hypersecretion, gastritis, colic, motion sickness, short bowel syndrome, a bowel dysfunction, radiation-induced injury to the gastrointestinal tract, chronic sore throat, a noncardiac chest pain, coughing, dysphagia, Shwachman syndrome, decreased gastric mucin production, iron deficiency anemia, decreased nasal airflow, pancreatitis, or cystic fibrosis.

3. The method of claim 1, wherein the psychiatric disorder is obsessive-compulsive disorder, schizophrenia, a schizoaffective disorder, depression, mania, manic-depression, apathy, delirium, a phobia, amnesia, or an eating disorder.

4. The method of claim 1, wherein the learning disability is attention deficit hyperactivity disorder, autism, dyslexia, mental retardation, developmental delay, or Shwachman syndrome.

5. The method of claim 1, wherein the sleep disorder is sleep apnea, a REM disorder, sleep onset with depression, an age-related sleep disorder, narcolepsy, sleep deprivation or a REM-deprived sleep disorder.

6. The method of claim 1, further comprising administering a therapeutically effective amount of one or more compounds selected from the group consisting of a proton pump inhibitor; a histamine antagonist; an antacid; a bismuth compound; VIP or an analog and/or fragment thereof; secretin or an analog and/or fragment thereof; sucralfate; cisapride; misoprostol; a vitamin supplement; an NSAID; a steroid; a cholinergic compound; a cholinesterase inhibitor; an anti-psychotic compound; a mood stabilizer; an anti-anxiety compound; a stimulant; an antidepressant; estrogen; a serotonin antagonist; a calcium channel blocker; a beta-adrenergic blocker; and an anticonvulsant compound.

7. The method of claim 1, wherein the compound of Formula (I), (II), (III), (IV), (V), (VI), (VII), (VIII), (IX), (X), (XI) or (XII) is administered in an amount of 0.01 to 1,000 mg per day.

8. The method of claim 1, wherein the compound of Formula (I), (II), (III), (IV), (V), (VI), (VII), (VIII), (IX), (X), (XI) or (XII) is administered in an amount of 0.1 to 500 mg per day.

9. The method of claim 1, wherein the compound of Formula (I), (II), (III), (IV), (V), (VI), (VII), (VIII), (IX), (X), (XI) or (XII) is administered in an amount of 0.1 to 100 mg per day.

10. The method of claim 1, wherein the compound of Formula (I), (II), (III), (IV), (V), (VI), (VII), (VIII), (IX), (X), (XI) or (XII) is administered orally.

11. The method of claim 1, wherein the compound of Formula (I), (II), (III), (IV), (V), (VI), (VII), (VIII), (IX), (X), (XI) or (XII) is administered topically.

12. The method of claim 1, wherein the compound of Formula (I), (II), (III), (IV), (V), (VI), (VII), (VIII), (IX), (X), (XI) or (XII) is administered nasally.

13. The method of claim 1, wherein the compound of Formula (I), (II), (III), (IV), (V), (VI), (VII), (VIII), (IX), (X), (XI) or (XII) is administered by injection.

14. The method of claim 13, wherein the compound of Formula (I), (II), (III), (IV), (V), (VI), (VII), (VIII), (IX), (X), (XI) or (XII) is administered in an amount of 1 to 3000 μg/kg.

15. The method of claim 13, wherein the compound of Formula (I), (II), (III), (IV), (V), (VI), (VII), (VIII), (IX), (X), (XI) or (XII) is administered in an amount 3 to 1000 μg/kg.

16. The method of claim 1, wherein the compound of Formula (I), (II), (III), (IV), (V), (VI), (VII), (VIII), (IX), (X), (XI) or (XII) is in the form of a pharmaceutically acceptable salt; a stereoisomer; or a pharmaceutically acceptable salt of a stereoisomer.
